(19) United States
(12) Patent Application Publication
(10) Pub. No.: US 2008/0021339 A1
(43) Pub. Date: Jan. 24, 2008

(54) Anesthesia monitor, capacitance nanosensors and dynamic sensor sampling method

(76) Inventors: Jean-Christophe P. Gabriel, Pinole, CA (US); Vikram Joshi, Newington, CT (US); John Loren Passmore, Berkeley, CA (US); Sergei Skarupo, Berkeley, CA (US); Alexander Star, Pittsburgh, PA (US); Christian Valcke, Orinda, CA (US)

Correspondence Address: O''Melveny & Myers LLP;IP&T Calendar Department LA-1118, 400 South Hope Street, Los Angeles, CA, 90071-2899, US

(21) Appl. No.: 11/588,845

(22) Filed: Oct. 26, 2006

Related U.S. Application Data
(63) Continuation-in-part of application No. 11/488,456, filed on Jul. 18, 2006.
(60) Provisional application No. 60/730,905, filed on Oct. 27, 2005.
(60) Provisional application No. 60/773,138, filed on Feb. 13, 2006.
(60) Provisional application No. 60/748,834, filed on Dec. 9, 2005.

(51) Int. Cl. A61B 5/08; G01N 31/22

(52) U.S. Cl. 600/532; 422/57

(57) ABSTRACT

Embodiments of nanoelectronic sensors are described, including sensors for detecting analytes such as anesthesia gases, CO2 and the like in human breath. An integrated monitor system and disposable sensor unit is described which permits a number of different anesthetic agents to be identified and monitored, as well as concurrent monitoring of other breath species, such as CO2. The sensor unit may be configured to be compact, light weight, and inexpensive. Wireless embodiments provide such enhancements as remote monitoring. A simulator system for modeling the contents and conditions of human inhalation and exhalation with a selected mixture of a treatment agent is also described, particularly suited to the testing of sensors to be used in airway sampling.

### CROSS-REFERENCE TO RELATED APPLICATION

This application claims priority pursuant to 35 USC. § 119(e) to the following US Provisional Applications, each of which applications are incorporated by reference:
No. 60/730,905 filed Oct. 27, 2005, entitled “Nanoelectronic Sensors And Analyzer System For Monitoring Anesthesia Agents And Carbon Dioxide In Breath”
No. 60/850,217 filed Oct. 6, 2006, entitled “Electrochemical nanosensors for biomolecule detection”;
No. 60/773,138 filed Feb. 13, 2006 entitled “Nanoelectronic Capacitance Sensors For Monitoring Analytes”;
No. 60/748,834 filed Dec. 9, 2005 entitled “Nanoelectronic Sensors Having Substrates With Pre-Patterned Electrodes, And Environmental Ammonia Control System”.

This application is a continuation-in-part of and claims priority to U.S. patent application Ser. No. 11/488,456 filed Jul. 18, 2006 (published 2006-______) entitled “Improved Carbon Dioxide Nanosensor, And Respiratory CO2 Monitors” which is incorporated by reference; and which in turn claims the priority to the following U.S. provisional and non-provisional patent applications, each of which applications are incorporated by reference:
Ser. No. 11/437,275 filed May 18, 2006 (published 2006-______) entitled “Nanoelectronic Breath Analyzer and Asthma Monitor”
Ser. No. 11/390,493 filed Mar. 27, 2006 (published 2006-______) entitled “Nanoelectronic Measurement System For Physiologic Gases, And Improved Nanosensor For Carbon Dioxide”
Ser. No. 11/111,121 filed Apr. 20, 2005 (published 2006-0055,392) entitled “Remotely communicating, battery-powered nanostructure sensor devices”
Ser. No. 11/019,792 filed Dec. 18, 2004 (published 2005-0245,836) entitled “Nanoelectronic capnometer adapter”
Ser. No. 10/940,324 filed Sep. 13, 2004 (published 2005-0129,573) entitled “Carbon Dioxide Nanoelectronic Sensor”
Ser. No. 10/656,898 filed Sep. 5, 2003 (published 2005-0279,987) entitled “Polymer Recognition Layers For Nanostructure Sensor Devices”
No. 60/700,944 filed Jul. 20, 2005
No. 60/683,460 filed May 19, 2005
No. 60/665,153 filed Mar. 25, 2005
No. 60/564,248, filed Apr. 20, 2004
No. 60/531,079 filed Dec. 18, 2003
No. 60/502,485 filed Sep. 12, 2003
No. 60/408,547 filed Sep. 5, 2002

This application is related in subject matter to U.S. patent application Ser. No. 11/090,550 filed Mar. 25, 2005 entitled “Sensitivity Control For Nanotube Sensors”, which is a divisional of Ser. No. 10/280,265 filed Oct. 26, 2002 (U.S. Pat. No. 6,894,359), which claims priority to U.S. Patent No. 60/408,412 filed Sep. 4, 2002; each of which applications are incorporated by reference.

This application is related in subject matter to U.S. patent application Ser. No. 10/846,072 filed May 14, 2004 (published 2005-0184,641) entitled “Flexible Nanotube Transistors”, which claims priority to U.S. No. 60/471,243 filed May 16, 2003; each of which applications are incorporated by reference.

This application is related in subject matter to U.S. patent application Ser. No. 10/177,929 filed Jun. 21, 2002 entitled “Dispersed Growth Of Nanotubes On A Substrate” (equivalent published as WO04-040,671); each of which applications are incorporated by reference.

This application is related in subject matter to U.S. patent application Ser. No. 11/139,184 filed May 27, 2005 entitled “Modification Of Selectivity For Sensing For Nanostructure Device Arrays”, which is a continuation of Ser. No. 10/388,701 filed Mar. 14, 2003 (U.S. Pat. No. 6,905,655), which claims the priority of U.S. Patent No. 60/366,566 filed Mar. 22, 2002, and which also is a continuation-in-part of U.S. Ser. No. 10/099,664 filed Mar. 15, 2002; each of which applications are incorporated by reference.

### BACKGROUND OF THE INVENTION

1. Field of the Invention

The present invention relates to nanoelectronic devices, and in particular to nanostructured sensor systems for measurement of medically relevant species, such as anesthetic agents in a patient's breath.

2. Description of Related Art

Medical breath analysis and monitoring may employ measurements of many chemical species to improve diagnosis and patient care. In general, exhaled breath has a composition which is distinct from inspired air. Compounds are either removed from inspired air (e.g., oxygen as O2 is absorbed and metabolized) or added to exhaled breath (e.g., CO2, H2O). The primary constituents of exhaled breath include N2, O2, CO2, water vapor and other atmospheric constituents (e.g., argon and the like).

Treatment compounds (e.g., anesthetic agents, anti-inflammatory agents, and the like) may be added to inspired air for inhaled administration, and the concentrations of such treatment compounds in breath may be monitored to assess patient condition. In addition, many volatile organic and inorganic chemical species which are produced by metabolic processes within the body are released in exhaled breath (some in only trace amounts). For example, nitric oxide (NO), nitrogen dioxide (NO2), other nitrogen-containing compounds, sulfur-containing compounds, alcohol, hydrogen peroxide, carbon monoxide, hydrogen, ammonia, ketones, aldehydes, esters, alkanes, and other volatile organic compounds may be present in exhaled breath. Metabolic breath species often have medical significance and may pertain to various conditions, including tissue inflammation, immune responses, metabolic and digestive processes, liver, kidney and heart problems, and other physiological conditions. Thus, sensitive and selective measurements of breath species must be made in the context of complex breath composition.

The measurement of inspired and end-tidal level of anesthesia agent is an important parameter to monitor for the anesthesiologist since it is the only direct indication available for the uptake of anesthesia agent by the patient. Indeed, one cannot only rely on the amount of agent delivered by the anesthesia machine since the percentage of uptake agent can vary considerably from patient to patient.

Today, state-of-the-art anesthesia measurement is based on non-dispersive infrared (IR) detection. The measurement apparatus typically costs over $5,000 (e.g., Datex-Ohmeda Div. of Instrumentarium Corp., Helsinki, Finland) and requires daily calibration, costly in human resources, time and calibration gases. A slipstream sample of respiratory gas is extracted from the anesthesia machine loop and fed through small diameter tubing to the IR detector. The tubing itself is subject to plugging by mucus or water condensation and the IR detector requires a condenser to avoid water droplets in the IR cell. Additionally, 100 mL/min of waste airflow must be vented to the environment. All together, the sampling and detection apparatus adds a bulky and cumbersome presence in the surgical suite.

Various agents have been used for anesthesia. Probably the most widely spread is nitrous oxide, (N₂O), which is used extensively in surgical operations. Analytical instruments based on conventional infrared spectrophotometry and photoacoustic spectroscopy have been available for the measurement of N₂O as well as mass spectrometers. Development of a metal oxide semiconductor N₂O sensor has been described in the literature. See, for example, E Kanazawa et al, “Metal oxide semiconductor N2O sensor for medical use,” Sens. Actuators B (2001), n77, pp 72-77, which is incorporated by reference. However, None of these approaches offer the required selectivity for the simple field measurement envisioned herein.

The other most common inhalation anesthetic agents are halogenated compounds. The use of enflurane and halothane in clinical anesthesia has either disappeared or declined in the US, due mainly to their significant toxicities. Newer less soluble fluorinated ethers are now used preferably, (isoflurane, sevoflurane and desflurane), because they are less soluble, have less side effects and have more desirable properties. See, for example, A B Dobkin, Ed, “Development of New Volatile Inhalation Anesthetics,” Elsevier, 1979, which is incorporated by reference. In addition to concentration monitoring, Real-time ability to automatically identify and distinguish anesthetic agents, is desirable to prevent situations where the anesthetic agent may be administered from the wrong vaporizer.

In the US, general anesthesia is administered more than 30 million times per year. If anesthesia gas monitoring could be done using a no-maintenance low-cost disposable sensor instead of a high maintenance, high operation cost and expensive IR detectors, significant savings would result. With even a modest cost savings per operation, very large savings per year that could be realized. Moreover, a less expensive sensor will enable small clinics to perform surgeries with improved monitoring, sot that the impact on patient safety will be significant. Indeed, it is common in third world countries to rely solely on the mechanical settings of the vaporizer. In addition, this technology represents one more step in the miniaturization of medical equipment, an important factor (i) for all aid workers and agencies that have strong weight and space contingencies when setting up field hospitals; (ii) in space constrained environment such as scans area (CT-scan, MRI).

The measurement of carbon dioxide or “CO2” concentration in the breath, can provide complementary advantages to the measurement of anesthesia agents, especially when available in an integrated device. (Note: where no ambiguity is created and for consistency with current patent database formats, chemical formulas are generally written herein with numbers in normal text, rather than subscripts or superscripts. Likewise, variables conventionally particularized by subscripts are denoted with lower case normal text)

The measurement of carbon dioxide levels in respiration is a standard procedure during intensive care and anesthesia and is a primary tool in the diagnosis and management of respiratory function. CO2 detection in breath has been used as an indicator of perfusion and heart function as well as ventilator effectiveness. In addition, CO2 is useful, by itself or in combination with other measurements, in diagnosing and monitoring airway status and pulmonary function. For example, see U.S. Pat. No. 6,648,833 entitled “Respiratory analysis with capnography”, which is incorporated by reference.

In the measurement of the variation profile of carbon dioxide (CO2) concentration in the breath, sometimes referred to as capnography, prevailing technology relies on bulky and expensive non-dispersive infrared absorption (NDIR) sensors to determine CO2 concentration. The high cost, complexity, weight and other limitations of this technology restrict the use of capnography to high value, controlled environments, such as surgical wards. This limits the medical use of capnography.

If anesthesia and CO2 measurement are integrated, it will allow continuous monitoring of the patient (from CT-scanner to surgery areas for example). This would improve patient safety and also save cost associated with work flow efficiency. Currently only the electrocardiogram, the pulse oximetry and the blood pressure are monitored during transport. In addition, data collection should not be interrupted and sequential (an uninterrupted stream of data during all phases of patient care), eliminating manually recorded data.

Thus, lower cost, simplified and integratable devices for the monitoring of anesthesia agents will greatly improve patient care in places and countries were limited funds for health care do not allow for monitoring level of anesthesia gases in surgical procedures and where the anesthesiologist must rely on the mechanical settings of the vaporizer, a risky situation for patient safety. It is also desirable to eliminate the constant gas diversion used for sampling, a true closed-loop anesthesia system can conserve costs, heat and eliminate emissions.

### SUMMARY OF THE INVENTION

Anesthesia agent monitors based on nanoelectronic sensors having aspects of the invention, such as nanotube-based capacitance and transistor devices, provide a device to inexpensively identify and measure concentrations of anesthesia agents in patient breath, allow surgical procedures to be more cost-effective, save setup time and allow more cost-effective delivery of medical care in places and countries were limited funds for health care do not allow for monitoring level of anesthesia gases in surgical procedures and where the anesthesiologist must rely on the mechanical settings of the vaporizer, a risky situation for patient safety.

Exemplary embodiments of nanoelectronic sensors having aspects of the invention have a conductive (e.g., semiconducting) nanostructured element, the nanostructured element comprising a nanostructured material. The nanostructured material may include one or more nanotubes or the like (e.g., nanorods, nanowires; and/or nanoparticles). In certain embodiments, a nanostructured material may comprise a film, mat, array or network of nanotubes or the like. The nanostructured element may be configured to include a layer, coating or channel, and may be disposed adjacent a substrate or support structure. Nanostructured materials comprising a nanostructured element may be non-functionalized, or may functionalized to alter properties. In some embodiments, a nanoelectronic sensor may include a recognition material, layer or coating disposed in association with the nanostructured element, wherein the recognition material may be configured to influence the response of the sensor to an analyte of interest (e.g., increase sensitivity, response rate, or the like) and/or may be configured to influence the response of the sensor to the operating environment (e.g., increase selectivity, reduce interference or contamination, or the like).

A preferred nanostructured material for employment in nanoelectronic sensors is the carbon nanotube. Nanotubes were first reported in 1993 by S Iijima and have been the subject of intense research since. Single walled nanotubes (SWNTs) are characterized by strong covalent bonding, a unique one-dimensional structure, and exceptionally high tensile strength, high resilience, metallic to semiconducting electronic properties, high current carrying capacity, and extreme sensitivity to perturbations caused by charged species in proximity to the nanotube surface.

Nanoelectronic sensor embodiments provide a large sensing surface in a tiny, low-power package which can directly sample and selectively monitor anesthesia agent concentrations. A single sensor chip may include a plurality of sensors, capable of measuring multiple anesthetic agents, such as N2O, Isoflurane, sevoflurane and desflurane which are currently the most commonly used agents in the USA, as well as CO2 and other breath species. Much of the signal processing may be built into the sensor board, requiring only simple and inexpensive external instrumentation for display and data logging, so as to provide a fully calibrated, sterilized, packaged and disposable anesthesia gas sensor. The small size of the nanoelectronic sensors permit them to fit directly in an anesthesia system airway, so as to avoid cumbersome tubing, condenser, pump, and exhaust system currently required to perform sampling.

Alternative embodiments having aspects of the invention include systems configured to include multiplexed assays on a single sensor platform or chip, microprocessors and/or wireless transceivers, permitting convenient recordation and analysis of patient-specific measurement histories and/or remote patient monitoring by treatment personnel. The output is digital so electronic filtering and post processing may be used to eliminate extraneous noise, if need be. See, for example, U.S. patent application Ser. No. 11/111,121 filed Apr. 20, 2005 entitled “Remotely communicating, battery-powered nanostructure sensor devices”; which is incorporated by reference.

Alternative embodiments having aspects of the invention are configured for detection of analytes employing nanostructured sensor elements configured as one or more alternative types of electronic devices, such as capacitive sensors, resistive sensors, impedance sensors, field effect transistor sensors, and the like, or combinations thereof. Two or more such measurement strategies in a may be included in a sensor device so as to provide orthogonal measurements that increase accuracy and/or sensitivity. Alternative embodiments have functionalization groups or material associated with the nanostructured element so as to provide sensitive, selective analyte response.

Although in the description herein a number of exemplary sensor embodiments are based on one or more carbon nanotubes, it is understood that other nanostructured materials known in the art may also be employed, e.g., semiconductor nanowires, various form of fullerenes, multiwall nanotubes, and the like, or combinations thereof. Elements based on nanostructures such carbon nanotubes (CNT) have been described for their unique electrical characteristics. Moreover, their sensitivity to environmental changes (charged molecules) can modulate the surface energies of the CNT and be used as a detector. The modulation of the CNT characteristic can be investigated electrically by building devices that incorporate the CNT (or CNT network) as an element of the device. This can be done as a conductive transistor element or as a capacitive gate effect.

Certain exemplary embodiments having aspects of the invention include single-walled carbon nanotubes (SWNTs) as semiconducting or conducting elements. Such elements may comprise single or pluralities of discrete parallel NTs, e.g., in contact or electrically communicating with a device electrode. For many applications, however, it is advantageous to employ semiconducting or conducting elements comprising a generally planar network region of nanotubes (or other nanostructures) substantially randomly distributed adjacent a substrate, conductivity being maintained by interconnections between nanotubes.

One embodiment of a sensor having aspects of the invention comprises an NTFET transistor device comprising nanostructured element having a sensitivity to nitrous oxide (N2O).

One embodiment of a sensor having aspects of the invention comprises a conductive nanostructured element configured to interact with an analyte of interest so as to alter at least one electrical property of the nanostructured element. The sensor comprises one or more electrodes and suitable circuitry configured to obtain measurements for at least one of capacitance, transconductance, resistance, impedance, transistor characteristics, or the like, in response to the exposure of the sensor to a sample, and to employ the measurements to determine the presence or concentration of an analyte of interest, such as an anesthesia agent.

One embodiment of a sensor having aspects of the invention comprises a processor configured to apply an algorithm relating at least two measured properties of a nanostructured element responsive to exposure to a sample, the relationship of the properties (e.g., a ratio change of resistance to change of capacitance) to determine the presence or concentration of an analyte of interest.

One embodiment of a sensor having aspects of the invention comprises a capacitance circuit in which at least one capacitive element includes a network or film comprising conductive nanostructured material (“nanostructured network”—e.g., an interconnecting network of semiconducting carbon nanotubes), wherein the capacitive element is configured to interact with a sample, wherein the circuit configured to respond to the presence of an analyte of interest by a measurable change in an electrical property.
(a) In certain embodiments, a capacitance sensing circuit comprises at least a pair of spaced-apart capacitive elements, in which both elements comprise a nanostructured network, and at least one capacitive element is configured to interact with a sample, wherein the circuit configured to respond to the presence of an analyte of interest by a measurable change in an electrical property.
(b) In certain embodiments, a capacitance circuit device comprises at least a pair capacitive structures arranged in series, such that at least one measurement may be made of an electrical property of the combined pair of structures (e.g., an impedance). For example, the capacitance circuit device may be configured as a sensor, wherein the measured property may be employed, at least in part, to determine the presence or concentration of an analyte when the device is exposed to a sample.
(c) In an alternative embodiment of (b), a connection between the pair of capacitive structures includes an electrically continuous nanostructured network.
(d) In an alternative embodiment of (c), an electrically continuous nanostructured network is configured to form both a connection between the pair of capacitive structures, and at least a portion of a capacitive element of each capacitive structure. For example, a nanostructured network may be disposed to cover and span between two adjacent spaced-apart conductive plates, the network being isolated from each plate by a dielectric layer.

One embodiment of a method having aspects of the invention comprises the steps of selectively exposing a sensor to a sample (e.g., delimiting sensor exposure by means of fluidic lumens and valves), and dynamically sampling a signal output from the sensor (e.g., delimiting signal to selected response ranges, time intervals and the like), so as to determine the presence or concentration of an analyte of interest by analysis of the dynamically sampled signal. Advantageously, the sensor may be exposed to a sample environment only intermittently without reducing the effective real-time monitoring of an analyte in the environment (e.g., the sensor exposure may be sequenced by an automatic fluidic sampling system). Furthermore, the physio-chemical impacts of the environment upon the sensor may be substantially reduced, without reducing the effective real-time monitoring of an analyte in the environment (e.g., sensor service life may be extended).

### BRIEF DESCRIPTION OF THE DRAWINGS

The following is a list which summarizes the drawings and figures herein:

FIG. 1 is a cross-sectional diagram which illustrates an exemplary electronic sensing device for detecting an analyte, configured in this example as a NTFET.

FIG. 2 are photographic views of a sensor system such as shown in FIG. 1, wherein views (a-c) include SEM images showing (a) showing the layout of interdigitated source and drain contacts S, D, (b) showing an enlarged detail of a nanotube network N and the contacts S, D, and (c) showing an enlarged detail of the margin of network N. View (d) shows an example of a sensor device mounted in a conventional electronic device package.

FIG. 3 is a cross-sectional diagram which illustrates an exemplary electronic sensing device, similar in a number of respects to the device of FIG. 1, configured in this example configured for measurement of capacitance and related properties as a signal for detecting an analyte, such as a fluorinated organic anesthetic agent.

FIG. 4 illustrates an alternative nanosensor embodiment having aspects of the invention, configured for measurement of breakdown voltage and related properties.

FIG. 5 illustrates an alternative nanosensor embodiment having aspects of the invention, configured for measurement of a signal based on electrochemical reactions involving an analyte of interest.

FIG. 6 is a series of five molecular diagrams of medically important fluorinated organic anesthetic agents.

FIG. 7 is plot showing the response of a device generally similar to that of FIG. 5 to brief sequential impingement of gas analyte samples (in air) containing first isoflurane and second halothane.

FIGS. 8A-8C are plots showing the responses of a device generally similar to those of FIGS. 1-3 (including circuitry for measurement of both source-drain resistance and source-gate capacitance) to sequential samples of a selected anesthetic agent gas in air, through a graded series of concentrations, in which:

FIG. 8A shows the response of a capacitance signal to samples sevoflurane in air;

FIG. 8B shows the response of a capacitance signal to samples isoflurane in air;

FIG. 8C shows the response of a capacitance signal to samples halothane in air;

FIGS. 9A-9D are plots showing the responses of a device general similar to that of FIGS. 8A-8C, in which:

FIG. 9A shows the response of both capacitance signal resistance signals to samples sevoflurane in air;

FIG. 9B shows the response of both capacitance signal resistance signals to samples isoflurane in air;

FIG. 9C shows the response of both capacitance signal resistance signals to samples halothane in air; and

FIG. 9D graphically illustrates the relative ratios of change of resistance and capacitance for 5% concentration of each agent in air, as depicted in FIGS. 9A, 9B and 9C.

FIG. 10 is plot showing the response in the channel current signal of a device generally similar to that of FIG. 1-2 to air only, and to a mixture of nitrous oxide (N₂O) in air.

FIGS. 11A-11C are plots showing transconductance response of a device generally similar to that of FIG. 1 and functionalized for CO₂ detection, wherein:

FIG. 11A shows the relative conductance through a large dynamic range of 500 to 10⁵ ppm of CO₂ in air,

FIG. 11B shows the channel current in response to a series of concentrations of CO₂ in air ranging from 500 to 10,000 ppm (0.05% to 1%), and

FIG. 11C shows the calibrated CO₂ percent concentration in response to breathing inhalation and exhalation of an exemplary electronic capnography system including sensor devices sensor generally similar to those of FIGS. 1-2.

FIG. 12 shows an exemplary integrated breath analysis system having aspects of the invention including an airway connector/sampler.

FIGS. 13A-B is a diagrammatic depiction of an alternative configuration of a portable medical gas sensing system comprising an integrated CO2 sensing, O2 delivery cannula, shown connected to a processor/input/display unit.

FIGS. 14A-B are a cross-section and top view respectively of the integrated CO2 sensing, O2 delivery cannula, included in FIGS. 13A-B.

FIG. 15 is a plan-view diagram including view (a) which illustrates an exemplary planar nanotube capacitor sensor device, and view (b) which is an enlarged detail of the sensor structure.

FIG. 16 is a plan view, cross-sectional view, and equivalent circuit diagram of an exemplary capacitive nanosensor embodiment having aspects of the invention, comprising a bi-layer architecture.

FIG. 17 is a plan view, cross-sectional view, and equivalent circuit diagram of an exemplary capacitive nanosensor embodiment having aspects of the invention, comprising off-set capacitor elements in series.

FIG. 18 is a cross-sectional view and a magnified portion of an exemplary capacitive nanosensor embodiment having aspects of the invention, generally similar to that shown in FIG. 17 and having a multi-layer dielectric structure.

FIG. 19 is a schematic and equivalent circuit diagram which illustrates an exemplary capacitive nanosensor embodiment having aspects of the invention, and having a bi-layered architecture comprising a porous substrate supporting CNT network “plates” with off-set contact regions, wherein view A-D show sequential plan views in suggested order of assembly, and view XC shows a cross section.

FIG. 20 is a schematic and equivalent circuit diagram which illustrates an exemplary capacitive nanosensor embodiment having aspects of the invention, comprising off-set capacitor elements in series, disposed in a “small gap” interdigitated arrangement, wherein view A-C show sequential plan views in suggested order of assembly, and view D shows a cross section.

FIGS. 21 and 22 are cross-sectional views showing exemplary nanostructured devices having a network element such as a CNT network which is electrically coupled to multiple leads without direct lead-to-network contact.

FIG. 23 is a schematic plot illustrating principles of a dynamic sensor sampling method having aspects of the invention.

FIG. 24 is a schematic plot an example of dynamic sensor sampling for a step change in analyte concentration, having a fixed maximum response cut-off values and a fixed recovery interval.

FIG. 25 is a schematic plot an example of dynamic sensor sampling for a step change in analyte concentration, having both fixed maximum and minimum response cut-off values.

FIG. 26 is a schematic plot an example of dynamic sensor sampling for a step change in analyte concentration, having a both fixed measurement and recovery intervals.

### DETAILED DESCRIPTION OF THE PREFERRED EMBODIMENT

Exemplary Nanosensor Architecture

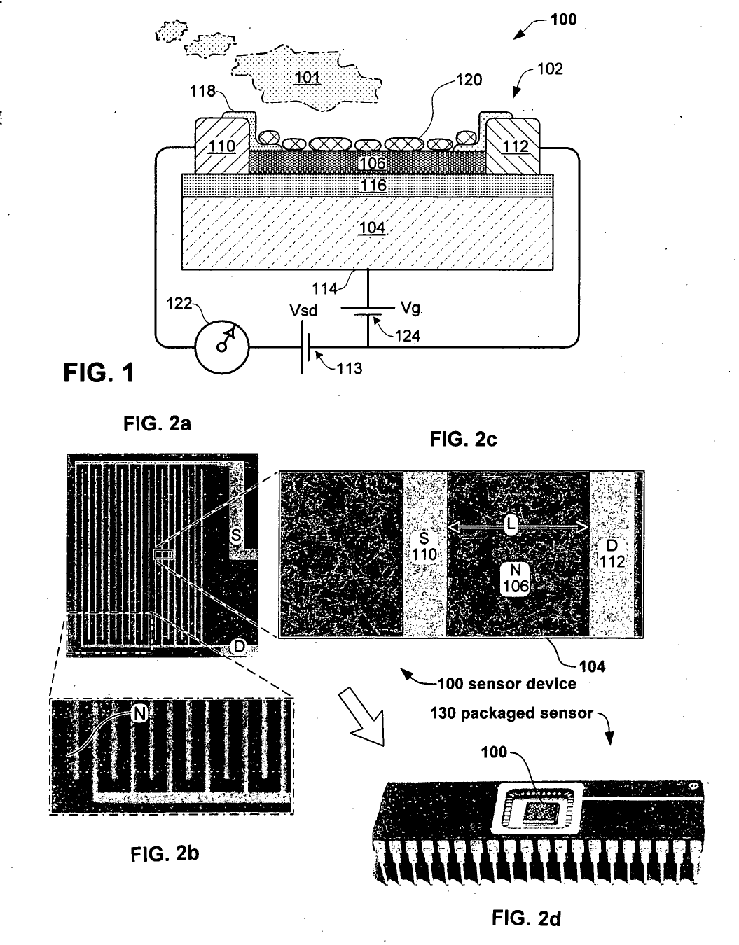

FIG. 1. shows an exemplary electronic sensing device 100 having aspects of the invention, for detecting an analyte 101 (e.g. CO₂, N₂O, isoflurane, halothane, sevoflurane, and the like). A number of alternative sensor device architectures and operating modes are possible, and may be employed alone or in combinations without departing from the spirit of the invention. In the example of FIG. 1, sensing device 100 includes a nanostructure sensor 102 configured for convenient transconductance measurements, as well as other properties. Sensor 102 comprises a substrate 104.

Sensor 102 comprises a conductive nanostructured element configured to include a channel, coating or layer 106 and comprising a nanostructured material (e.g., one or more conducting or semiconducting nanotubes, nanorods, nanowires and/or nanoparticles; a film, mat or network of nanotubes; combinations of these; or the like). The nanostructured element (layer or channel 106) may be disposed adjacent the substrate 104. Channel or layer 106 may contact the substrate as shown, or in the alternative, may be spaced a distance away from the substrate, with or without a layer of intervening material. In a preferred embodiment, layer 106 comprises an interconnecting network including a plurality of semiconducting single-walled carbon nanotubes (SWNTs).

One or more conductor elements, contacts or electrodes 110, 112 may be disposed over the substrate and electrically connected to channel or layer 106.

Elements 110, 112 may comprise metal electrodes in contact with conducting channel 106. In the alternative, a conductive or semi-conducting material (not shown) may be interposed between contacts 110, 112 and conducting channel 106. Contacts 110, 112 may comprise source and drain electrodes, respectively, upon application of a source-drain voltage Vsd. The voltage or polarity of source 110 relative to drain 112 may be variable, e.g., the applied voltage may be DC, AC, pulsed, or variable. In an embodiment of the invention, the applied voltage is a DC voltage.

In an embodiment of the invention, conducting channel 106 may comprise a plurality of carbon nanotubes forming a mesh, film or network. Such a network may be formed by various suitable methods. One suitable approach may comprise forming an interconnecting network of single-wall carbon nanotubes directly upon the substrate, such as by reacting vapors in the presence of a catalyst or growth promoter disposed upon the substrate. For example, single-walled nanotube networks can be grown on silicon or other substrates by chemical vapor deposition from iron-containing catalyst nanoparticles with methane/hydrogen gas mixture at about 900 degree C. Advantageously, the use of highly dispersed catalyst or growth-promoter for nanostructures permits a network of nanotubes of controlled diameter and wall structure to be formed in a substantially random and unclumped orientation with respect to one another, distributed substantially evenly at a selected mean density over a selected portion of the substrate.

Alternatively, a nanotube network may be deposited on a device substrate by spray deposition and the like. For example, single wall carbon nanotubes (SWNTs) and/or other nanoparticles may be suspended in a suitable fluid solvent, and sprayed, printed or otherwise deposited in a substrate. The SWNTs or other nanoparticles may optionally have additional functionalization groups, purification and/or other pre-deposition processing. For example SWNTs functionalized with poly m-aminobenzene sulfonic acid (PABS) show hydrophilic properties and may be dispersed in aqueous solutions.

One or more conductive traces or electrodes may be deposited after deposition, or alternatively, the substrate may include pre-patterned electrodes or traces exposed on the substrate surface. Similarly, alternative embodiments may have a gate electrode and a source electrode supported on a single substrate. The substrate may include a flat, sheet-like portion, although one skilled in the art will appreciate that geometric variations of substrate configurations (rods, tubes or the like) may be employed without departing from the spirit of the inventions.

The density of a network of nanotubes (or other nanostructure elements) may be adjusted to achieve a selected conductivity in an electrically continuous network via interconnections between adjacent nanotubes (e.g., a CNT film of density close to but greater than the percolation limit). For example, this may be achieved through controlled CVD conditions (e.g., catalyst particle density, deposition environment, duration, or the like); by controlled flow through a filter membrane (see L. Hu et al., “Percolation in Transparent and Conducting Carbon Nanotube Networks”, Nano Letters (2004), 4, 12, 2513-17, which is incorporated by reference), by controlled deposition from a fluid carrier (e.g., spray deposition); or the like.

In a spray-deposition example, multiple light, uniform spray steps may be performed (e.g., with drying and resistance testing between spray steps) until the network sheet resistance reaches a target value (implying a target network density and conductivity). In one example, P2-SWNTs produced by Carbon Solutions, Inc of Riverside, Calif. were spray-deposited on a portion of a PET sheet substrate with pre-patterned traces until a sheet resistance about 1 kΩ was reached.

See also the methods for making nanotube networks as well as additional device and substrate alternatives as described the following patent applications, each of which is incorporated by reference:
U.S. patent application Ser. No. 10/177,929 filed Jun. 21, 2002 entitled “Dispersed Growth Of Nanotubes On A Substrate”, (PCT equivalent published as WO04-040,671);
U.S. application Ser. No. 10/846,072 filed May 14, 2004, entitled “Flexible nanotube transistors” (Publication 2005-0184,641);
U.S. patent application Ser. No. 11/274,747 filed Nov. 14, 2006 entitled “Nanoelectronic Glucose Sensors”; and
U.S. Patent Application No. 60/748,834, filed Dec. 9, 2005, entitled “Nanoelectronic Sensors Having Substrates With Pre-Patterned Electrodes, And Environmental Ammonia Control System”.

In addition to nanotube films or networks, films or other arrangements of other nanostructures, including individual nanostructures, can be used. Alternative nanostructures may include, for example, nanospheres, nanocages, nanococoons, nanofibers, nanowires, nanoropes and nanorods. Such alternative nanostructures may be adapted similarly to nanotubes for the embodiments described herein. Nanostructures can be made of many different elements and compounds. Examples include carbon, boron, boron nitride, and carbon boron nitride, silicon, germanium, gallium nitride, zinc oxide, indium phosphide, molybdenum disulphide, and silver.

In the example of FIG. 1, the device 100 may be operated as a gate-controlled field effect transistor, with sensor 102 further comprising a gate electrode 114. Such a device is referred to herein as a nanotube field effect transistor or NTFET. Gate 114 may comprise a base portion of substrate 104, such as a doped-silicon wafer material isolated from contacts 110, 112 and channel 106 by a dielectric layer 116, so as to permit a capacitance to be created by an applied gate voltage V_{g}. For example, the substrate 104 may comprise a silicon back gate 114, isolated by a dielectric layer 116 comprising SiO₂. Alternatively gate 114 may include a separate counter electrode, liquid gate or the like.

Sensor 102 may further comprise a layer of inhibiting or passivation material 118 covering regions adjacent to the connections between the conductive elements 110, 112 and conducting channel 106. The inhibiting material may be impermeable to at least one chemical species, such as to the analyte 101 or to environmental materials such as water or other solvents, oxygen, nitrogen, and the like. The inhibiting material 118 may comprise a passivation material as known in the art, such as silicon dioxide, aluminum oxide, silicon nitride, or other suitable material. Further details concerning the use of inhibiting materials in a NTFET are described in prior co-invented U.S. Pat. No. 6,894,359 entitled “Sensitivity Control For Nanotube Sensors” which is incorporated by reference herein.

Device 100 may further comprise suitable circuitry in communication with sensor elements to perform electrical measurements. For example, a conventional power source may supply a source drain voltage V_{sd} (113) between contacts 110, 112. Measurements via the sensor device 100 may be carried out by suitable measurement circuitry represented schematically by meter 122 connected between contacts 110, 112. In embodiments including a gate electrode 114, a conventional power source 124 may be connected to provide a selected or controllable gate voltage V_{g}. Device 100 may include one or more electrical supplies and/or a signal control and processing unit (not shown) as known in the art, in communication with the sensor 102.

Optionally, device 100 may comprise a plurality of sensors like sensor 102 disposed in a pattern or array, such as described in prior application Ser. No. 10/388,701 filed Mar. 14, 2003 entitled “Modification Of Selectivity For Sensing For Nanostructure Device Arrays” (now published as US 2003-0175161), which is incorporated by reference herein. Each device in the array may be functionalized with identical or different functionalization. Identical device in an array can be useful in order to multiplex the measurement to improve the signal/noise ratio or increase the robustness of the device by making redundancy. Different functionalization may be useful for providing differential sensitivity so as to permit measurement of a profile of different responses to analytes.

The substrate 104 may be insulating, or on the alternative, may comprise a layered structure, having a base 114 and a separate dielectric layer 116 disposed to isolate the contacts 110, 112 and channel 106 from the substrate base 114. The substrate 104 may comprise a rigid or flexible material, which may be conducting, semiconducting or dielectric. Substrate 104 may comprise a monolithic structure, or a multilayer or other composite structure having constituents of different properties and compositions. For example, in an embodiment of the invention, the substrate 104 may comprise a silicon wafer doped so as to function as a back gate electrode 114. The wafer being coated with intermediate diffusion barrier of Si₃N₄ and an upper dielectric layer of SiO₂. Optionally, additional electronic elements may be integrated into the substrate for various purposes, such as thermistors, heating elements, integrated circuit elements or other elements.

In certain alternative embodiments, the substrate may comprise a flexible insulating polymer, optionally having an underlying gate conductor (such as a flexible conductive polymer composition), as described in application Ser. No. 10/846,072 filed May 14, 2004, which application is incorporated by reference. In further alternative embodiments, the substrate may comprise a polymeric substance coated with nanotube or other nanostructure particles in the in the manner described in U.S. application Ser. No. 11/274,747 filed Nov. 14, 2005, which application is incorporated by reference.

The conducting channel 106 (e.g., a carbon nanotube layer) may be functionalized to produce a sensitivity to one or more target analytes 101. Although nanostructures such as carbon nanotubes may respond to a target analyte through charge transfer or other interaction between the device and the analyte, more generally a sensitivity can be achieved by employing a recognition material 120, also called a functionalization material, that induces a measurable change in the device characteristics upon interaction with a target analyte. In addition or in substitution to the metallic nanoparticle functionalization, of the exemplary embodiments described in detail herein, the functionalization may alternatively include metal oxides, metal salts, polymers, and the like. Likewise, functionalization may include composite nanoparticles, mixtures of materials or the like.

In the exemplary embodiments described in detail herein, the recognition material disposed upon the channel 106 comprises on or more metallic materials. In particular, alternative embodiments of arrays of sensors such as shown in FIG. 1 may be functionalized with a range of materials different catalytic metals to produce cross-sensitive NTFET sensor elements.

FIG. 2 are photographic views (a-d) of a sensor system 100 such as shown in FIG. 1, wherein views (a-c) include SEM images showing (a) showing the layout of interdigitated source and drain contacts S 110 and D 112, (b) showing an enlarged detail of a nanotube network N 106 and the contacts S 110 and D 112, and (c) showing an enlarged detail of the margin of network N 106. View (d) shows an example of a sensor device 100 mounted in a conventional electronic device package 130. Note that the extent of a carbon nanotube network may be conveniently controlled by selective or masked oxidation of nanotubes from peripheral regions of the substrate 104 (“ashing”).

The conducting channel 106 (e.g., a carbon nanotube layer) may be functionalized to produce a sensitivity to one or more target analytes 101. Although nanostructures such as carbon nanotubes may respond to a target analyte through charge transfer or other interaction between the device and the analyte, a specific sensitivity may be achieved by employing a recognition material 120, also called a functionalization material, that induces a measurable change in the device characteristics upon interaction with a target analyte.

Device 100 may be packaged in a conventional manner to conveniently permit connection to operating circuitry. FIG. 2, view (d) is a photograph of a sensor device 100 generally similar to that of views (a-c), fabricated on a die of a wafer, and mounted as a chip in a conventional 40 pin CERDIP package using wirebonding techniques. Device 100 may further comprise suitable circuitry in communication with sensor elements to perform electrical measurements. For example, a conventional power source may supply a source-drain voltage (Vsd) between contacts 110, 112. Measurements via the sensor device 100 may be carried out by circuitry represented schematically by meter 122 connected between contacts 110, 112. In embodiments including a gate electrode 114, a conventional power source 124 may be connected to provide a selected or controllable gate voltage (Vg). Device 100 may include one or more electrical supplies and/or a signal control

Alternative Nanosensor Architectures.

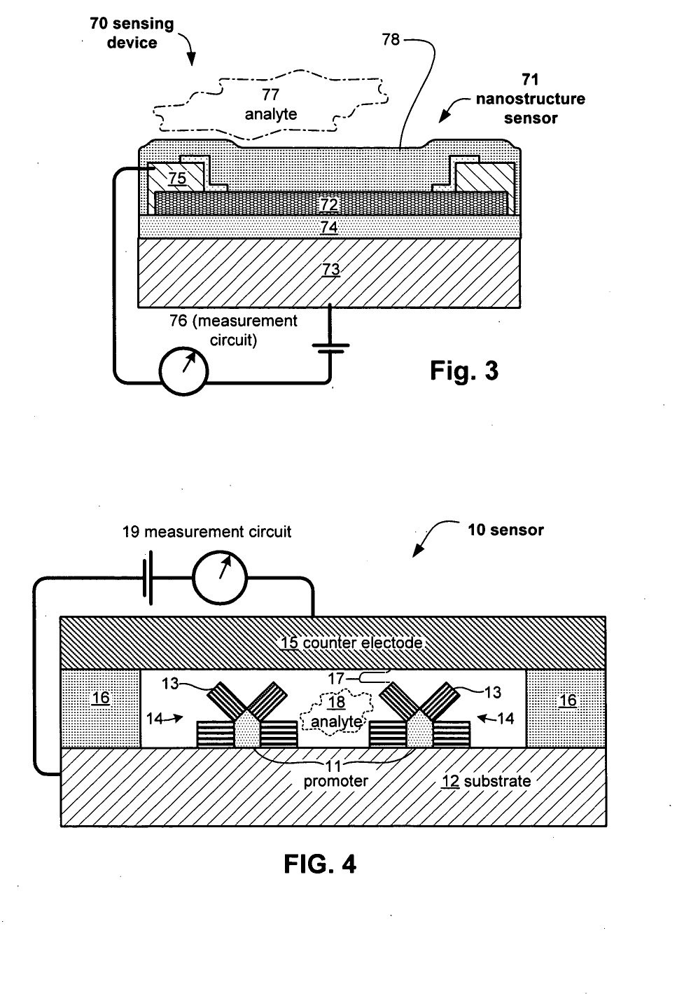

FIG. 3 shows one example of a an exemplary electronic sensing device 70 having aspects of the invention, similar in a number of respects to the device of FIG. 1, configured in this example as a capacitance sensor for detecting an analyte, as further described in commonly invented and assigned U.S. Provisional Application No. 60/773,138 filed Feb. 13, 2006; No. 60/660,441, filed Mar. 10, 2005; and No. 60/669,126, filed Apr. 6, 2005, each of which is incorporated by reference. Nanostructured capacitance sensors are particularly effective for detecting species such as fluorinated organic anesthetic agents.

As shown in FIG. 3, Sensor device 70 includes a nanostructure sensor 71 which includes a conductive nanostructured element configured to include a channel, coating or layer 72 comprising a nanostructured material (see description of example of FIG. 1). In an exemplary embodiment, the nanostructured material includes a carbon nanotube network 72, disposed upon a substrate comprising a dielectric isolation layer 74 disposed upon a base 73, in this example a doped silicon wafer back gate.

The nanotube network 72 is contacted by at least one conductive electrode 75 (a pair are shown, in this case having optional passivation on the electrode-nanotube contact region). A conditioning/recognition structure 78 may be included, disposed adjacent network 72, and may included functionalization or recognition material, analyte conditioners (e.g., a filter, selectively permeable polymer, etc.) and the like.

The sensor device 70 further includes at least a capacitance measurement circuit 76 in electrical communication with contact 75 and back gate 73, so as to permit the capacitance and/or impedance of the spaced apart nanotube network/back gate assembly to be readily measured (i.e., the total charge required to be placed on either conductor to create a given voltage potential between conductors, C=Q/V).

It should be understood that other capacitor conductors may be substituted for back gate 73 or added to the device 70 without departing from the spirit of the invention, such as a top gate, liquid gate, a second spaced-apart nanotube network conductor, and the like. Additionally, many alternative functional arrangements of the respective conductors are possible. The capacitance C of the sensor 71 may be calibrated, and compared analytically with the capacitance during exposure to analyte of interest 11 (e.g., isoflurane, halothane, and the like). In particular, species having significant dipole moments may act to change the capacitance upon interaction with the nanotube network 72.

As shown in FIG. 3, additional functionalization 78 may be included in sensor 71 (e.g., an absorbent filter, a selectively permeable polymer layer, a selectively reactive or binding species, etc., to enhance selectivity, sensitivity and/or signal strength). See, for example, U.S. Provisional Application No. 60/669,126, filed Apr. 6, 2005, entitled “Systems Having Integrated Cell Membranes And Nanoelectronics Devices, And Nano-Capacitive Biomolecule Sensors”, which is incorporated by reference.

FIG. 4 illustrates principles of alternative sensors suitable for measurement of anesthetic agents, as well as other analytes, employing measurements related to characteristic “breakdown voltage” and/or the self-sustaining discharge current. In the example of FIG. 4, perpendicularly aligned CNTs may be grown on a SiO2 growth promoter, delimited by adjacent surfaces of growth inhibiting materials, such as exposed Si wafer substrate or vapor deposited metals, such as Au. See, for example, B. Q Wei et al, “Organized Assembly of Carbon Nanotubes”, Nature (2002) Vol 416, pp 495-496; and A Modi et al, “Miniaturized Gas Ionization Sensors Using Carbon Nanotubes”, Nature (2003) Vol 424 pp 171-174. Each of these publications is incorporated by reference.

As shown in FIG. 4, discharge breakdown nanosensor 10 includes nanotube electrodes spaced apart from a conductive electrode. In this example, the sensor comprises one or more regions of a CNT growth promoter 11 (e.g., SiO2 islands) disposed on a substrate 12 (e.g., doped Si wafer). CNT material 13 is grown using CVD upon the promoter material, preferably being formed generally perpendicularly to the promoter surface to a controlled length. Preferably the promoter is shaped (by known methods) so as to cause at least a portion of the CNT material to be deposited at an angle to the substrate. The CNT material formation 14 functions as an electrode, and more preferably as an anode.

The sensor further comprises a counter electrode formation 15, preferably a cathode, comprising in this example a metallic plate (e.g. aluminum) set apart from the substrate by spacers 16 (e.g. spacers comprising an insulator or material having an insulating coating) so as to establish a selected gap 17 between the CNT anode 14 and the metallic cathode 15. In certain embodiments, the sensor comprises a plurality of CNT anodes of a generally similar height, and the spacers are configured to position the counter electrode generally parallel to the substrate. Openings (not shown) are provided for communication of an analyte medium 18 to the gap region 17.

Electrical measurement circuitry 19 of conventional design is connected to the anode and cathode. In one mode of operation, a DC voltage is applied between the anode and cathode so as to create an electric field across the gap. In this example, the CNT anodes are electrically connected via electrical communication or contact with conductive substrate. Due to the nanoscale tip radius, and due to the small region of CNT material adjacent the gap (“gap” in this case referring to the minimum distance between anode and cathode), the field gradient is very high near the CNT tips, resulting in a corona of ionized gas of the analyte medium. The medium experiences a “breakdown” or avalanche of emitted electrons at a voltage with a function of the constituent species of the analyte medium. Preferably, the measurement circuitry includes measurement components (not shown) for temperature and pressure for purposes of calibration. The breakdown voltage and/or the self-sustaining discharge current may be characterized by the measurement circuitry, so as to identify constituent species and/or their concentration in the medium.

FIG. 5 shows schematic architecture of a sensor device embodiment 50 having aspects of the invention for detection and measurement of analyte species, for example, by detection of electrochemical energy associated with the presence of an analyte. The device 50 comprises a sensor substrate 52 (e.g., comprising PET, polycarbonate, flexible polymers, or the like) having a reaction or sensor tip portion of its surface 60 on which an interconnecting carbon nanotube (CNT) network 54 is disposed. In the example of FIG. 5, a conductive trace or drain 55 electrically communicates with the network 54 (e.g., silver ink may be deposited on the substrate 12 so as to contact a portion of the network 54). Device 50 includes a well or container 57 holding buffer or fluid media 59 in which both sensor tip 60 and a gate electrode 58 are immersed. In certain embodiments, gate electrode 58 may include a reference electrode, such as a Ag/AgCl reference electrode, saturated calomel electrode, or the like. One skilled in the art will appreciate that container 57 may comprise one or more microfluidic elements, capillaries, sampling devices, incubators, and the like, without departing from the spirit of the invention.

An encapsulation material 56 (e.g., polymers such as epoxy, Al2O3, Si4N3, SiO2, ALD layers, and the like) may be deposited so as to isolate portions of the device from the medium or buffer 59, while not covering at least a portion of the CNT network 54. With reference to encapsulation material 56 and to other encapsulation layers, dielectric layers and/or isolation layers or multi-layer structures included in alternative embodiments having aspects of the invention described herein, it may be advantageous to produce layers that are extremely thin and uniform, while at the same time avoiding pores, shadowing or other discontinuities/irregularities in the coating. It may also be desirable in certain elements to avoid damage to underlying elements, such as carbon nanotube networks. Atomic layer deposition methods provide alternative approaches to producing a layer or coating having these desirable qualities, and may be employed to deposit a layer of an oxide, nitride or other compound, or combinations or multiple layers of these.

Alternative methods may be used, such as thermal and e-beam evaporation. Additional process elements may be included to improve coating properties, such as rotating and/or tilting a substrate during evaporation. Further description of ALD methods may by found in P. Chen, et al, “Atomic Layer Deposition to Fine-Tune the Surface Properties and Diameters of Fabricated Nanopores”, Nano Lett (June 2004) Vol. 4, No. 7, pp 1333-37; D. Farmer et al, “Atomic Layer Deposition on Suspended Single-Walled Carbon Nanotubes via Gas-Phase Noncovalent Functionalization”, Nano Lett (March 2006) Vol. 6, No. 4, pp 699-703; and M. Groner et al, “Gas diffusion barriers on polymers using Al2O3 atomic layer deposition”, Appl. Phys. Lett. (2006) Vol. 88, pp 051907-1; which publications are incorporated by reference.

Drain 55 and gate 58 are connected to suitable measurement circuitry 53, which may comprise one or more of a number of devices conventionally used for signal measurement, recordation, display, power supply, signal processing and/or logic operations, and the like, as described further herein. Additional or substitute electrodes may also be included in device 50, such as counter electrodes, reference electrodes and the like, such as Ag/AgCl reference electrodes described herein.

Redox couple species may be included in detection media (e.g., ferrocyanide/ferricyanide redox couple) to enhance electron transfer between the media and the nanostructured electrode material, such as SWNTs. For example, a ferrocyanide/ferricyanide redox couple may include 10 mM solution of Fe(CN)63-/4- added to AP buffer. Such a redox couple may produce more than 100 fold increase of electron transfer between solution and the device as indicated by square voltammetry method.

Exemplary sensor device embodiments having aspects of the invention may include other electrodes in addition to a nanostructured recognition electrode. For example, a gate electrode, a reference electrode, a counter electrode, or the like may be included. Electrodes may be connected to suitable measurement circuitry and instruments conventionally used for signal measurement, recordation, display, power supply, signal processing logic operations, or the like. Detection may include measurement and comparisons of a variety of different electrical properties, including amphometric, transconductance and capacitance measurements; impedance spectroscopy; cyclic voltammetry; square wave voltammetry; or the like. Measurement methods are further described in A J Bard and L Faulkner, Electrochemical Methods: Fundamentals and Applications (Wiley and Sons, New York, 2001); and J Wang, Analytical Electrochemistry (Wiley and Sons, New York, 2000), which publications are incorporated by reference.

Further description of electrochemical sensors having aspects of the invention may be found in U.S. provisional application No. 60/850,217 filed Oct. 6, 2006, entitled “Electrochemical nanosensors for biomolecule detection”, which is incorporated by reference. One skilled in the art will recognize that analytes in a gas-phase sample may be detected following dissolution in a liquid medium. For example, sensor 50 may be configured so that fluid 59 is contained by a porous material, exposed to absorb analyte species from surrounding gaseous samples, such as exhaled human breath. In other alternative configurations, fluid 59 may be circulated via microfluidic channels, pumps and like components to absorb analyte species at a point relatively remote from sensor 50, the fluid 59 subsequently returned to be exposed to the sensor 50 for analyte detection.

Optionally, sensor devices having aspects of the invention (such as sensors 100, 70, 10 and 50 above) may be configured or disposed in a pattern or array, in various combinations. See for example, application Ser. No. 10/388,701, entitled “Modification Of Selectivity For Sensing For Nanostructure Device Arrays” (now U.S. Pat. No. 6,905,655), which is incorporated by reference herein. Each device in the array may be functionalized with identical or different functionalization. Identical device in an array can be useful in order to multiplex the measurement to improve the signal/noise ratio or increase the robustness of the device by making redundancy. Different functionalization may be useful for providing sensitivity to a greater variety of analytes with a single device. A sensor array embodiment may provide for a number of advantageous measurement alternatives, methods and benefits according to the invention, for example:
a) Multiple analytes detected by a plurality of specifically functionalized sensors,
b) Increased precision and dynamic range by a plurality of sensors each of which is optimized for a different range,
c) Increased analyte specificity and flexibility by detecting a characteristic “profile” of responses of a target analyte to a plurality of differently-functionalized sensors. Detection may be achieved by pattern recognition and “E-nose” methodology.
d) Self calibration systems and isolated reference sensors,
e) Multiple-use array having a plurality of deployable one-time-use sensor units,
f) patterned sample conditioning layers, materials or components may be applied to certain sensors of an array, while other sensors are exposed to unconditioned or differently-conditioned sample environments (e.g., selective filters, temperature gradients, pH gradients, redox couple reagents, bias fields, or the like); or
f) Ultra-low-cost, direct-digital-output sensor arrays, including a plurality of sensors, each producing a binary signal, and collectively having a range of response thresholds covering a selected analyte concentration range.

The electronic circuitry described is by way of illustration, and a wide range of alternative measurement circuits may be employed without departing from the spirit of the invention. Embodiments of a nanoelectronic sensor device having aspects of the invention may include an electrical circuit configured to measure one or more properties of the nanosensor, such as measuring an electrical property via the conducting elements.

Any suitable electrical property may provide the basis for sensor sensitivity, for example, electrical resistance, electrical conductance, current, voltage, capacitance, transistor on current, transistor off current, and/or transistor threshold voltage. In the alternative, or in addition, sensitivity may be based on a measurements including a combination of properties, relationships between different properties, or the variation of one or more properties over time. From such measurements, and from derived properties such as hysteresis, time constants, phase shifts, or scan rate/frequency dependence, correlations may be determined with target detection or concentration. The electronic sensor device may include or be coupled with a suitable microprocessor or other computer device as known in the art, which may be suitably programmed to carry out the measurement methods and analyze the resultant signals. Those skilled in the art will appreciate that other electrical or magnetic properties may also be measured as a basis for sensitivity. Accordingly, the embodiments disclosed herein are not meant to restrict the types of device properties that can be measured.

### Anesthesia Agent Sensor Examples

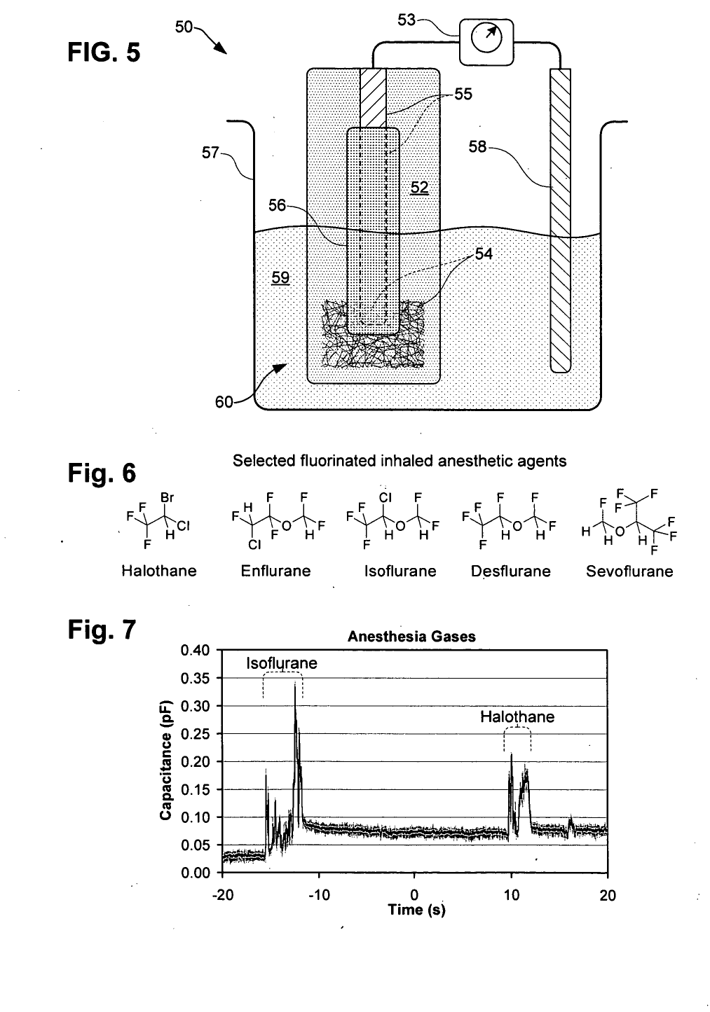

A considerable variety of compounds have been employed as anesthetic agents, including such organic and inorganic species as diethyl ether; nitrous oxide; chloroform; cyclopropane; trichloroethylene; fluoroxene; halothane; methoxyflurane; enflurane; isoflurane; desflurane; and sevoflurane. FIG. 6 shows a series of five molecular diagrams of medically important fluorinated organic anesthetic agents.

FIG. 7 shows a plot of the response of an exemplary nanostructure sensor to exposure to the anesthesia agents isoflurane and halothane, as further described in commonly invented and assigned U.S. Provisional Application No. 60/683,460, filed May 19, 2005, entitled “Multi-Valent Breath Analyzer Having Nanoelectronic Sensors, And Its Use In Asthma Monitoring”, which is incorporated by reference. The nanosensor employed is generally similar to that diagramed in FIGS. 1-3, and the plot shows the effect on a capacitance signal during a sequential exposure of the agents in the presence of ambient air, first a brief exposure to isoflurane, followed by a recovery period, and then subsequent exposure to halothane. The vertical axis is measured capacitance, and the horizontal axis is time in seconds. Note the reaction is very rapid, as is the recovery time. After the initial exposure, the recovered sensor capacitance is quite constant. In the example of FIG. 7, the nanotube network 72 of sensor 71 was directly exposed to the analyte media (air, with sample analyte admixed).

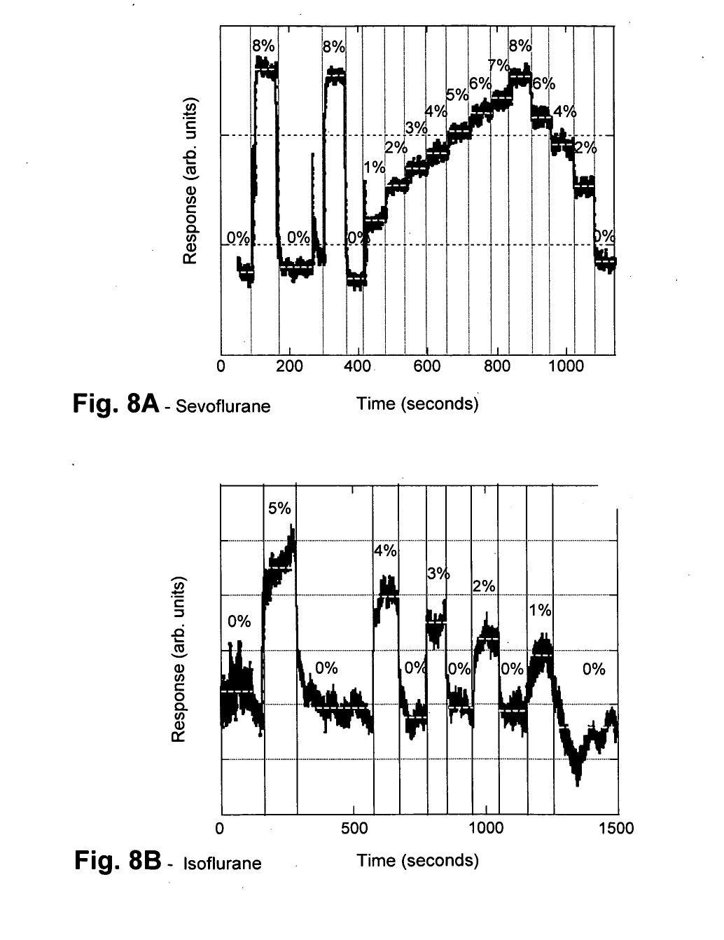

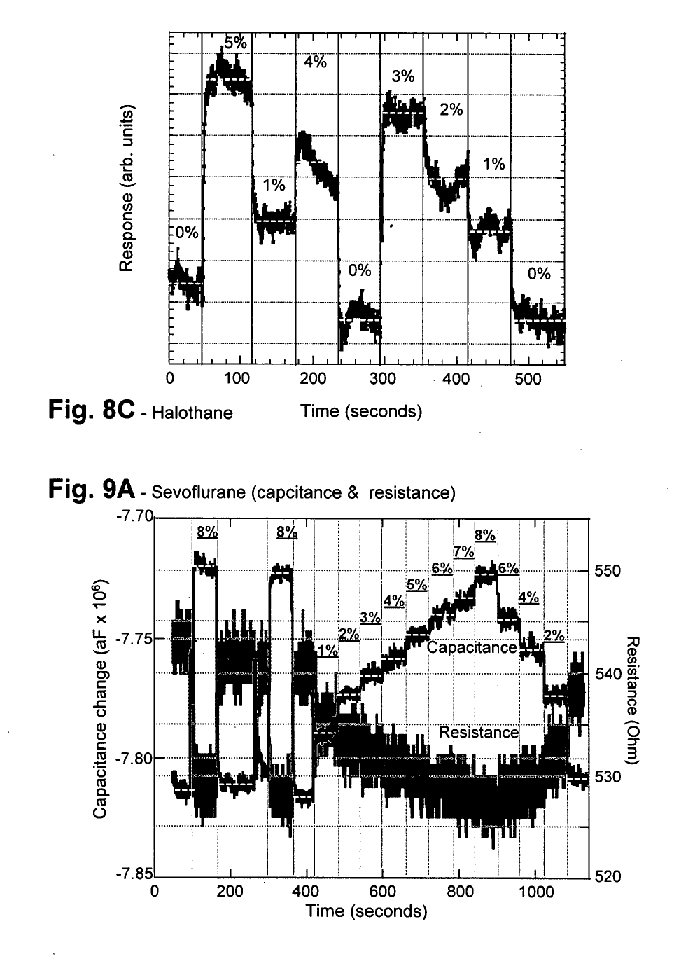

FIGS. 8A-8C are plots showing the responses of a device generally similar to those of FIGS. 1-3 (including circuitry for measurement of both source-drain resistance and source-gate capacitance) to sequential samples of a selected anesthetic agent gas in air, through a graded series of concentrations. The samples are administered in timed pulses of approximately 60 second duration each. The overlay dashed line at each concentration is not a measured value, but an approximated mean level, shown for clarity and convenience.

The sensors employed in the examples of FIGS. 8A-8C included a directly-exposed nanotube network, although various functionalization and conditioning layers or materials may optionally be included (see FIG. 3). CNT elements in devices without additional functionalization have been demonstrated to be generally not sensitive to gaseous CO2 in either resistance and capacitance measurements. In alternative examples, a selectively or partially permeable barrier material may be included to block exposure to water and other breath species while permitting anesthesia agents to diffuse to contact nanostructured elements such as a CNT network. Examples include Perfluorinated materials which may be deposited by spin coating or by chemical vapor deposition. Alternative materials include paraffin, Al2O3, polymers and the like. In addition, particular functionalization materials may be included to enhance sensitivity to particular analyte species (see functionalization material examples in Table 1 below).

FIG. 8A shows the response to samples sevoflurane in air. The sample pulses are administered in a graded series of concentrations ranging from 1% to 8% sevoflurane. The pulsed samples include of two initial cycles to the maximum concentration of 8%, separated by a comparable recovery period of air contact only. Like the data in FIG. 7, response and recovery seen in FIG. 8 are consistently very rapid, and return to consistent recovery capacitance level. Following the initial samples, the pulses proceed by graded steps, ramping increasing to maximum and then ramping decreasing to air-only. The response of capacitance is generally consistent between increasing and decreasing concentration, confirming the recovery performance.

FIG. 8B shows the response to samples isoflurane in air. The sample pulses are administered in a graded series of concentrations ranging from 1% to 5%, in most cases separated by a comparable recovery period of air contact only. Like the data in FIG. 8A, response and recovery consistently very rapid. The response of capacitance is generally consistent between increasing and decreasing concentration, and the recovery level is reasonably consistent.

FIG. 8C shows the response to samples halothane in air. The sample pulses are administered in a graded series of concentrations ranging from 1% to 5%, in most cases separated by a comparable recovery period of air contact only. The pattern of response is generally qualitatively similar the data in FIG. 8B, response and recovery consistently very rapid, the response of capacitance is generally consistent between increasing and decreasing concentration, and the recovery level is reasonably consistent.

FIG. 8C shows the response to samples halothane in air. The sample pulses are administered in a graded series of concentrations ranging from 1% to 5%, in most cases separated by a comparable recovery period of air contact only. The pattern of response is generally qualitatively similar the data in FIG. 8B, response and recovery consistently very rapid, the response of capacitance is generally consistent between increasing and decreasing concentration, and the recovery level is consistent.

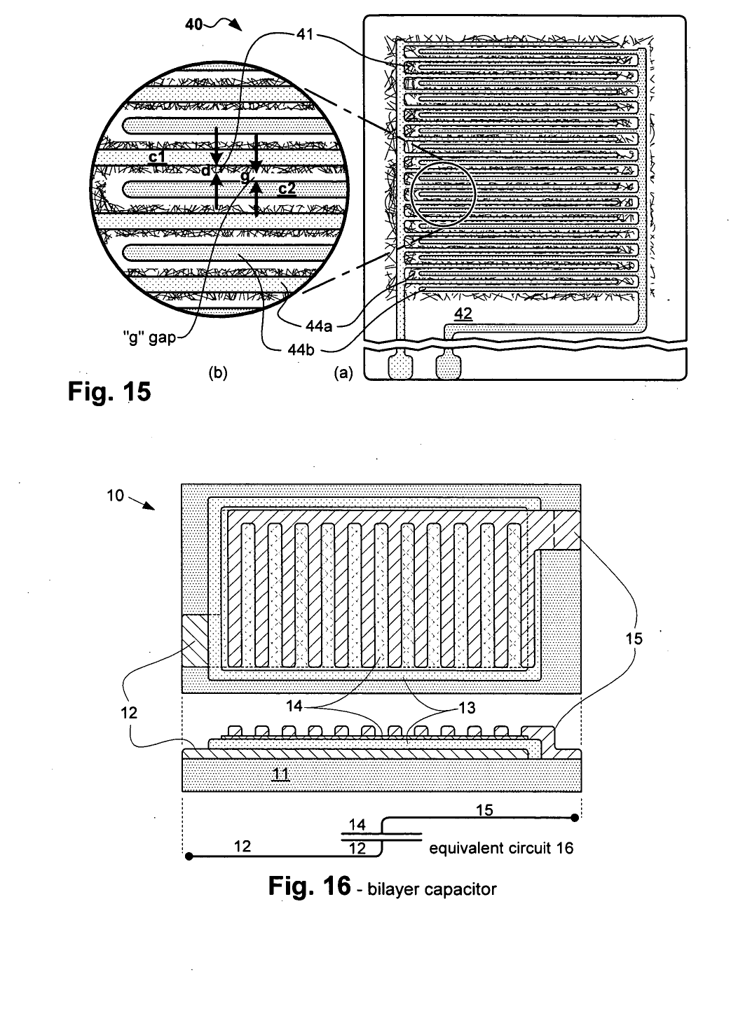

Simultaneous conductance and capacitance measurements on a nanostructure sensor element (e.g., a single-walled carbon nanotube (SWNT) network) may be used to extract an intrinsic property of molecular adsorbates. Measurements may be made of related properties as well, such as impedance of a sensor having a capacitive circuit architecture (see examples of FIGS. 15-22 below).

For example, adsorbed analytes from dilute chemical vapors produce a rapid response in both the capacitance and the conductance of a SWNT network. These responses are caused by a combination of two distinct physiochemical properties of the adsorbates: charge transfer and polarizability. It has been shown that the ratio of the conductance (or resistance) response to the capacitance response is a concentration-independent intrinsic property of a chemical vapor that can assist in its identification. See Eric S. Snow and F. Keith Perkins, “Capacitance and Conductance of Single-Walled Carbon Nanotubes in the Presence of Chemical Vapors”, Nano Lett (2005) 5 (12), 2414-2417, which publication is incorporated by reference.

Thus, a sensor system may produce a response which characterizes analyte identity in one output or signal analysis mode, and produce a response which characterizes analyte concentration in another output or signal analysis mode. In one exemplary embodiment having aspects of the invention, a sensor system may include capacitance and resistance measurement/processing circuitry communicating with a nanosensor (e.g., such as in FIG. 1) to determine the identity of an analyte employing a ratio of the resistance and capacitance change upon exposure to an analyte sample, and then determine a concentration of the thus-identified analyte from the capacitance change based on analyte-specific calibration data.

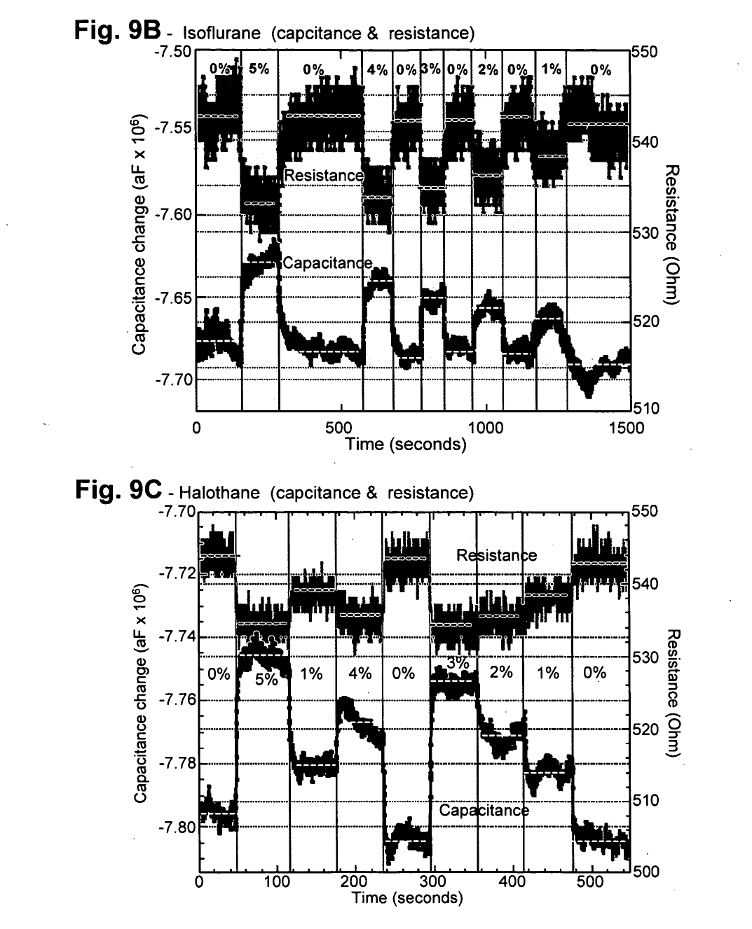

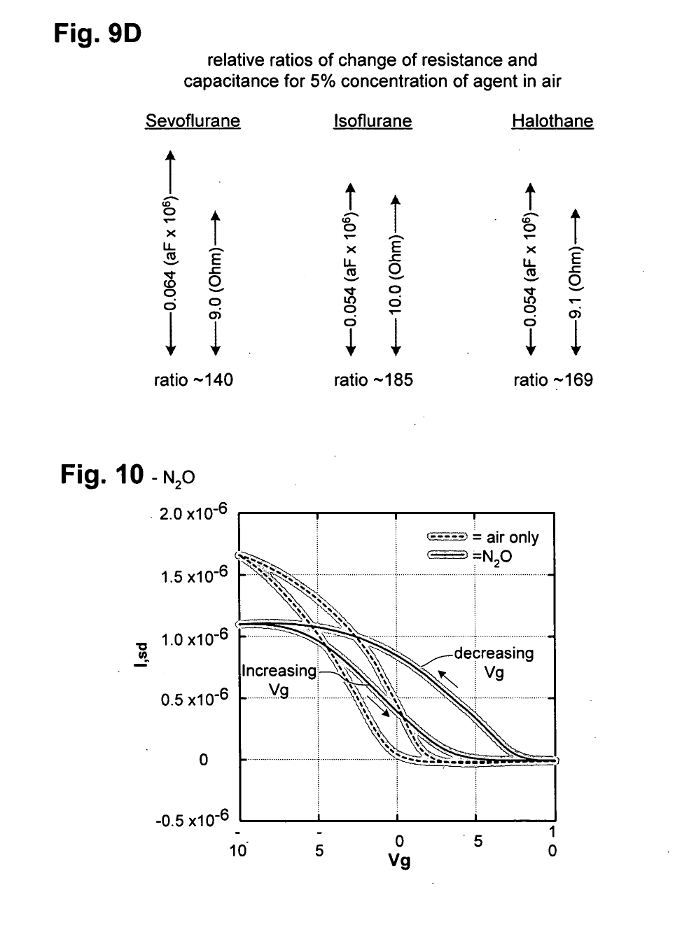

FIGS. 9A-9C are plots of the capacitance responses of the devices to agent exposures as shown in FIGS. 8A-8C, superimposed upon a signal measuring the simultaneous source-drain resistance, the capacitance units being shown on the left-hand axis, and the resistance units on the right-hand axis.

FIG. 9A shows the response of both capacitance signal resistance signals to samples sevoflurane in air. The response of the device to the agent in both the capacitance and resistance signals can be seen to be very rapid, with a rapid recovery. The relation of capacitance to sevoflurane concentration can be seen to be in the opposite direction, each generally proportional in magnitude to the other. FIGS. 9B and 9C show the responses of both capacitance signal resistance signals to samples isoflurane and halothane in air; respectively, plotted in the same manner as FIG. 9A.

FIG. 9D graphically illustrates the relative ratios of change of resistance and capacitance for 5% concentration of each agent in air, as depicted in FIGS. 9A, 9B and 9C. For each agent, the left arrow represents the magnitude of change of capacitance signal from air-only to an agent-air 5% mixture, and the right arrow represents the magnitude of the corresponding change in the resistance signal.

It can be seen from FIG. 9D that the ratio to the capacitance and resistance signals is a distinct value for each of the agents, sevoflurane, isoflurane and halothane. The this ratio may be used to confirm or distinguish the identity of an anesthetic agent, and advantageously this may be done in conjunction with the simultaneous measurement of the agent's concentration. Where Vg is the voltage of a substrate gate such as is shown in FIG. 1, the signals of capacitance and conductance (or resistance) may be converted for comparison (e.g., ratio calculation) to normalized values in units of ΔVg that represent the change in the substrate gate electrode (counter electrode) voltage required to produce an equivalent change in capacitance ΔC (or change in resistance ΔR), i.e. ΔC*=ΔC/(dC/dVg) and ΔG*=ΔR/(dR/dVg) where the derivatives are evaluated at Vg=0.

FIG. 10 is plot showing response in the channel current signal relative to variable gate voltage, of a device generally similar to that of FIGS. 1-2, upon exposure to air only, and to concentrated nitrous oxide (N2O). The nanotube network was functionalized with spin-coated polyimide. Note that additional or alternative functionalization materials may likewise be included (see Table 1 below). The exposure to N2O produces a marked decrease in maximum current (“on” current), and also shifts the threshold Vg to a higher voltage (curve shift to the right). Thus it may be seen that the NTFET provides a sensitive and specific measurement for N2O.

### Capnography (CO2) Sensor Examples

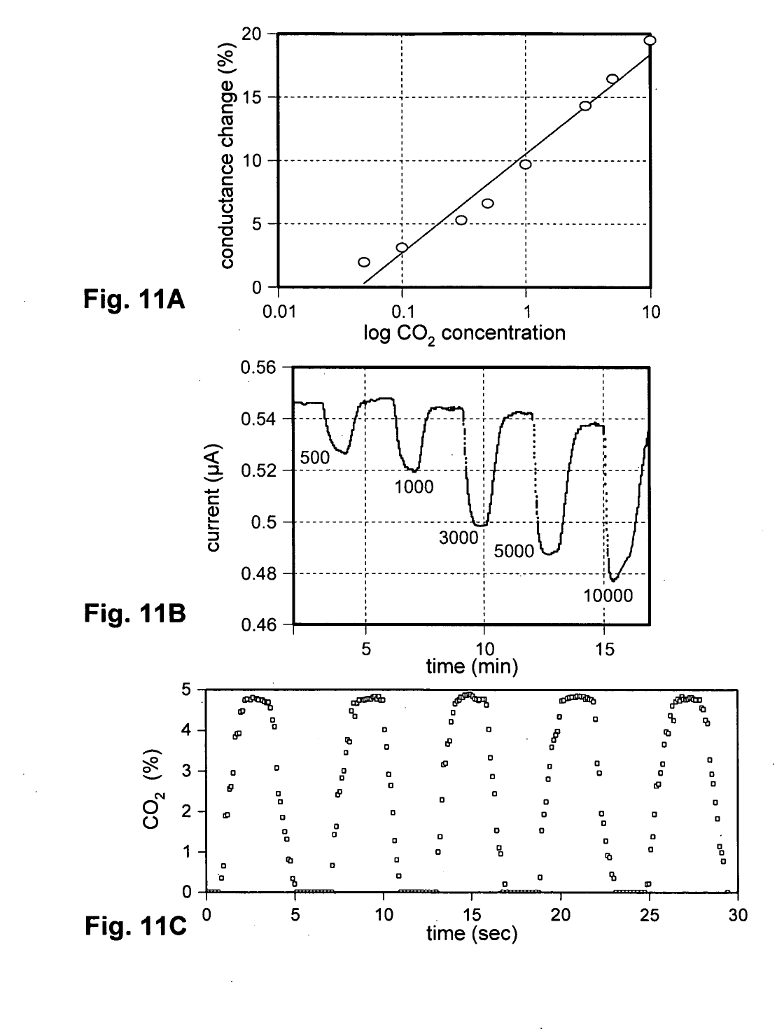

The measurement of breath CO2, separately or in conjunction with the measurement of anesthesia agents, may be employed as an important indicator of pulmonary and circulatory function. FIGS. 11A-11C illustrate the measurement of physiologic CO2 using nanosensors having aspects of the invention, as further described in commonly invented and assigned U.S. patent application Ser. No. 10/940,324 filed Sep. 13, 2004 entitled “Carbon Dioxide Nanoelectronic Sensor” (published 2005-0129,573); U.S. patent application Ser. No. 11/019,792 filed Dec. 18, 2004 entitled “Nanoelectronic Capnometer Adapter”; and U.S. patent application Ser. No. 11/488,456 filed Jul. 18, 2006 (published 2006-______) entitled “Improved Carbon Dioxide Nanosensor, And Respiratory CO2 Monitors”; each of which applications is incorporated by reference.

FIG. 11A is a plot showing transconductance response of an NTFET device generally similar to that of FIGS. 1-2 and functionalized for CO2 detection, and shows the relative conductance through a large dynamic range of 500 to 105 ppm of CO2 in air. FIG. 11A shows that the NTFET produces a substantially log response up to at least 10% CO2 in air, well beyond the range found in ordinary human breath. The recognition chemistry and specificity permit the sensor to operate at different relative humidities and shows low cross-sensitivity to oxygen, and to anesthesia gases, such as nitrous oxide and fluorinated organic agents.

In the exemplary embodiment, sensitivity to CO2 may be achieved using a suitable functionalization layer 120. The functionalization layer may perform two main functions: 1) to selectively recognize carbon dioxide molecules and 2) upon the binding of CO2 to generate an amplified signal that is transferred to the carbon nanotube transducer. In the presence of water, carbon dioxide forms carbonic acid which dissociates and alters the pH of the functionalization layer, thus protonating the electron donating groups and making the NTFET more p-type.

In an exemplary embodiment of a carbon dioxide (CO₂) sensor (see schematic of FIG. 1), sensitivity to CO₂ may be achieved using a suitable functionalization material or layer 120 (which may be continuous or discontinuous). The functionalization layer may perform two main functions: 1) to selectively recognize carbon dioxide molecules and 2) upon the binding of CO₂ to generate an amplified signal that is transferred to the carbon nanotube transducer. In the presence of water, carbon dioxide forms carbonic acid which dissociates and alters the pH of the functionalization layer, thus protonating the electron donating groups and making the NTFET more p-type. Basic inorganic compounds (e.g., sodium carbonate), pH-sensitive polymers, such as polyaniline, poly(ethyleneimine), poly(o-phenylenediamine), poly(3-methylthiophene), and polypyrrole, as well as aromatic compounds (benzylamine, naphthalenemethylamine, antracene amine, pyrene amine, etc.) may be used to functionalize NTFETs for CO₂ sensing. The functionalization layer may be constructed using polymeric materials such as polyethylene glycol, poly(vinyl alcohol) and polysaccharides, including various starches as well as their components amylose and amylopectin.

Functionalization material 120 may comprise more than one material and/or more than one layer of material, also referred to as “functionalization material”, “functionalization layer” or “functionalization”. The functionalization layer has two main functions: 1) it selectively recognizes carbon dioxide molecules and 2) upon the binding of CO2 it generates an amplified signal that is transferred to the nanostructure (e.g., carbon nanotube) transducer. Basic inorganic compounds (e.g., sodium carbonate), pH-sensitive polymers, such as polyaniline, poly(ethyleneimine), poly(o-phenylenediamine), poly(3-methylthiophene), and polypyrrole, as well as aromatic compounds (benzylamine, naphthalenemethylamine, anthracene amine, pyrene amine, etc.) can be used to functionalize NTFETs for CO2 sensing. The functionalization layer can be constructed using certain polymeric materials such as polyethylene glycol, poly(vinyl alcohol) and polysaccharides, including various starches as well as their components amylose and amylopectin. For example, a suitable reaction layer may be formed from a combination of PEI or similar polymer with a starch polymer. Other suitable materials for the functionalization layer may include, for example, metals, metal oxides, and metal hydroxides. In addition, a metallic functionalization layer may be combined with a polymeric functionalization layer.

Materials in the functionalization layer may be deposited on the NTFET using various different methods, depending on the material to be deposited. For example, inorganic materials, such as sodium carbonate, may be deposited by drop casting from 1 mM solution in light alcohols. The functionalized sensor may then be dried by blowing with nitrogen or other suitable drying agent. Polymeric materials may be deposited by dip coating. A typical procedure may involve soaking of the chip with the carbon nanotube device in 10% polymeric solution in water for 24 hours, rinsing with water several times, and blowing the chip dry with nitrogen. Polymers which are not soluble in aqueous solutions may be spin coated on the chip from their solutions in organic solvents. Values of polymer concentrations and the spin coater's rotation speeds may be optimized for each polymer.

In one exemplary embodiment having aspects of the invention, the functionalization layer 120 includes PAMAM or poly(amidoamine) dendrimer, which has a branched structure suitable for formation of hydrogels. PAMAM is available commercially in a number of types and forms, such as from Dendritic NanoTechnologies, Inc.; Dendritech, Inc; and Sigma-Aldrich Co. For example, an ethylenediamine core may have poly(amidoamine) branches with terminal amine groups. See Xu-Ye Wu, Shi-Wen Huang, Jian-Tao Zhang, Ren-Xi Zhuo, “Preparation and Characterization of Novel Physically Cross-linked Hydrogels Composed of Poly(vinyl alcohol) and Amine-Terminated Polyamidoamine Dendrimer”, Macromol. Biosci. 2004, 4, 71-75, which is incorporated by reference.

Functionalization material 120 may be comprised so as to balance hydrophobicity, hydrophilicity and basic properties (e.g., amino polymers), so as to optimize response time and cross-sensitivity to other species in the sample environment, such as relative humidity. The use of thin film coatings or assembled monolayers (SAM) can be employed to improve response time.

Alternative materials for layer 120 may include, for example, those shown in TABLE 1. Such materials may be included in sensors such as are describe herein without departing from the spirit of the invention.

TABLE 1
Examples of alternative recognition materials

| | |
|---|---|
| Polyacrylic acid | Polyurethane resin |
| Poly(acrylic acid-co-isooctylacrylate) | Polycarbazole |
| poly(ethylene imine), “PEI” | poly(sulfone) |
| poly(4-vinylphenol) | poly(vinyl acetate) |
| poly(alkyl methacrylate) | poly(vinyl alcohol) |
| poly(a-methylstyrene) | poly(vinyl butyral) |
| poly(caprolactone) | polyacrylamide |
| poly(carbonate bisphenol A) | polyacrylonitrile |
| poly(dimethylsiloxane) | polyaniline |
| poly(ethylene glycol) | polybutadiene |
| poly(ethylene oxide) | polycarbonate |
| poly(ethylenimine) | polyethylene |
| poly(methyl vinyl ether-co-maleic | polyoxyethylene |
| anhydride) | |
| poly(N-vinylpyrrolidone) | polypyrrole |
| poly(propylene) | polytetrafluoroethylene |
| poly(styrene) | polythiophene |
| polyvinyl-methyl-amine | Polyvinyl pyridine |
| polyaminostyrene | |
| chitosan | chitosan HCL |
| polyallylamine | polyallylamine HCL |
| poly(diallylamine) | poly(diallylamine) HCL |
| poly(entylene-co-vinyl acetate), | poly-(m-aminobenzene sulfonic |
| ˜82% ethylene | acid), “PABS” |
| poly(styrene-co-allyl alcohol), | poly(vinyl chloride-co-vinyl |
| ˜5.7% hydroxyl | acetate), ˜10% vinyl acetate |
| poly(styrene-co-maleic anhydride), | poly(vinylidene chloride-co- |
| ˜50% styrene | acrylonitrile), ˜80% vinylidene |
| | chloride |
| metalloporphyrin (M-porph) | Poly-L-lysine |
| Alpha-fetoprotein Profile Four (AFP4) | glycerol |
| Poly methyl methacrylate (PMMA) | polyglycerol |
| Nafion NR 50 | |
| metal coatings and nanoparticles: | Fe, V, Au, Pt, Pd, Ag, Ni, |
| | Ti, Cr, Cu, Mg, Al, Co,, Zn, |
| | Mo, Rh, Sn, W, Pb, Ir, Ru, Os, |
| | and alloys or mixtures |
| inorganic coatings and nanoparticles: | |
| V₂O₅ | WO₃ |
| Cu(SO₄) | Boric/Boronic acid |
| ZnO | Boron Trichloride |
| Fe₂O₃ | CaCl₂ |

Materials in the functionalization layer may be deposited on the NTFET using various different methods, depending on the material to be deposited. It should be understood that mixtures, alloys and composites of the materials may also be included. For many materials, ALD methodology is known which is suitable for depositing thin, uniform layers or coatings, which may be controlled to deposit on selected portions of a device, and which may be employed to produce mixtures or multi-layer coatings also. Other methods may be employed. For example, inorganic materials, such as sodium carbonate, may be deposited by drop casting from 1 mM solution in light alcohols. The functionalized sensor may then be dried by blowing with nitrogen or other suitable drying agent. Polymeric materials may be deposited by dip coating. A typical procedure may involve soaking of the chip with the carbon nanotube device in 10% polymeric solution in water for 24 hours, rinsing with water several times, and blowing the chip dry with nitrogen. Polymers which are not soluble in aqueous solutions may be spin coated on the chip from their solutions in organic solvents. Values of polymer concentrations and the spin coater's rotation speeds may be optimized for each polymer.

It is desirable in many respiratory medical applications to be able to detect breath CO2 concentration at a time resolution equal to a small fraction of the respiratory rate or breathing cycle. Accelerants or catalysts may be employed to improve the response of nanosensors having aspects of the invention. In one embodiment, catalysts are used to accelerate the conversion of CO2 in aqueous solution to carbonic acid. This reaction can produce an alteration in ambient pH so as to change a detectable property of the sensor, e.g. the conductance an carbon nanotube.

Such a sensor with may be employed for either or both of detection of CO2 in both gaseous or liquid sample media. For example, in one example of a respiratory sensor embodiment having aspects of the invention, an expired breath sample stream is directed into contact with a sensor package, and the following sequence of reactions may occur:
1. Gaseous CO² from the breath diffuses into a sensor structure containing water (e.g., H₂O bound in a hydrogel matrix adjacent a nanotube network): CO₂(g)←→CO₂(aq)
2. The CO2 reacts with water forming a carbonic acid: H₂O+CO₂(aq)←→H₂CO₃
3. The carbonic acid dissociates: H₂CO₃←→H⁺+HCO₃⁻
4. A cumulative modulation of pH occurs as the H⁺ increases to an equilibrium concentration: pH=−log [H⁺]
5. A change in a nanosensor electrical property is measured in response to modulation of pH (e.g., change in conductance of a nanotube network between a source and drain electrode pair).

Reaction 2. above is often found to be a rate-limiting step in sensor performance. It has been found that an enzyme such as carbonic anhydrase may be incorporated into a nanosensor as described herein, so as to accelerate the conversion of dissolved CO2 to carbonic acid. Such enzymes are important in living organisms to improve gas exchange processes. In one embodiment, a nanosensor comprises carbonic anhydrase is suspended in an appropriate buffer bound in a hydrogel matrix such as PAMAM, and disposed adjacent a carbon nanotube network. The effect of the enzyme can be dramatic: it has been found that the carbonic anhydrase can increase sensor response rate by 3 orders of magnitude or more, e.g., as measured by the rate of change of conductance of the nanotube network.

Further useful description may by found in J. Shin et al, “A Planar pCO2 Sensor with Enhanced Electrochemical Properties”, Anal. Chem. (2000), Vol. 72, pp 4468-73; which publication is incorporated by reference. Analogs to carbonic anhydrase, such as catalysts (e.g., Zn-[12]aneN3) may be similarly employed to increase the rate conversion of CO2 to carbonic acid by reaction 2 above. See for example the analogs and catalysts active on carbon dioxide as a substrate, as described in the following: G. Parkin, “Synthetic Analogues Relevant to the Structure and Function of Zinc Enzymes”, Chem. Rev. 2004, 104, 699-767; E. Kimura et al, “A Zinc(11) Complex of 1,5,9-Triazacyclododecane([12]aneN3) as a Model for Carbonic Anhydrase”, J. Am. Chem. Soc. 1990, 112, 5805-11; Joseph E Coleman, “Zinc enzymes”, review in “Bio-inorganic chemistry”, pp 222-234.

Similar principles may be usefully employed in nanosensors having aspects of the invention for the detection of such species as ammonia, nitric oxide, carbon monoxide, methane, and the like, to improve sensor response by enzymatic or catalytic acceleration of rate-limiting reaction steps.

For example, transition metal catalysts may be used to accelerate the conversion of breath NO to NO2 in a nanosensor having aspects of the inventions, wherein a sensor property is altered in the presence of NO2.

FIG. 11B is a plot showing the channel current response of an exemplary NTFET carbon dioxide sensor in response to a low range of low concentrations of carbon dioxide in concentrations of CO2 ranging from 500 to 10,000 ppm (0.05% to 1%). The response to CO2 gas is fast and reproducible at different concentrations. FIG. 11C shows the calibrated CO₂ percent concentration in response to breathing inhalation and exhalation of an exemplary electronic capnography system including sensor devices sensor generally similar to those shown in FIGS. 1-2. The performance of the sensor at this clinically relevant sensitivity range shows the great potential for these sensors in capnography and anesthesia medical applications.

As noted above, CO2 measurement is an important indicator of pulmonary and circulatory function. In particular time-domain measurements and profiles of the concentrations of breath species are medically useful indicators which have been correlated with particular medical conditions. For example, aspects of the measured profile of a patient's capnogram (the CO2 concentration in exhaled breath versus exhalation time) have been correlated with such conditions as bronchial spasms, asthma, obstructive lung disease, restrictive lung disease, and the like. It has also been demonstrated that the profile of a capnogram can be correlated with real-time expiratory flow rate and other spirometric parameters. See, for example, D Hampton et al, U.S. Pat. No. 6,648,833 entitled “Respiratory analysis with capnography”; B You et al, “Expiratory capnography in asthma: evaluation of various shape indices”, Eur Respir J (1994); 7(2) pp 318-23; M Yaron et al, “Utility of the expiratory capnogram in the assessment of bronchospasm”, Ann Emerg Med (1996) 28(4) pp 403-7; and B You et al, “Expiratory capnography in asthma. Perspectives in the use and monitoring in children”, Rev Mal Respir (1992) 9(5) pp 547-52; each of which publication is incorporated by reference.

Integrated Breath Analysis System

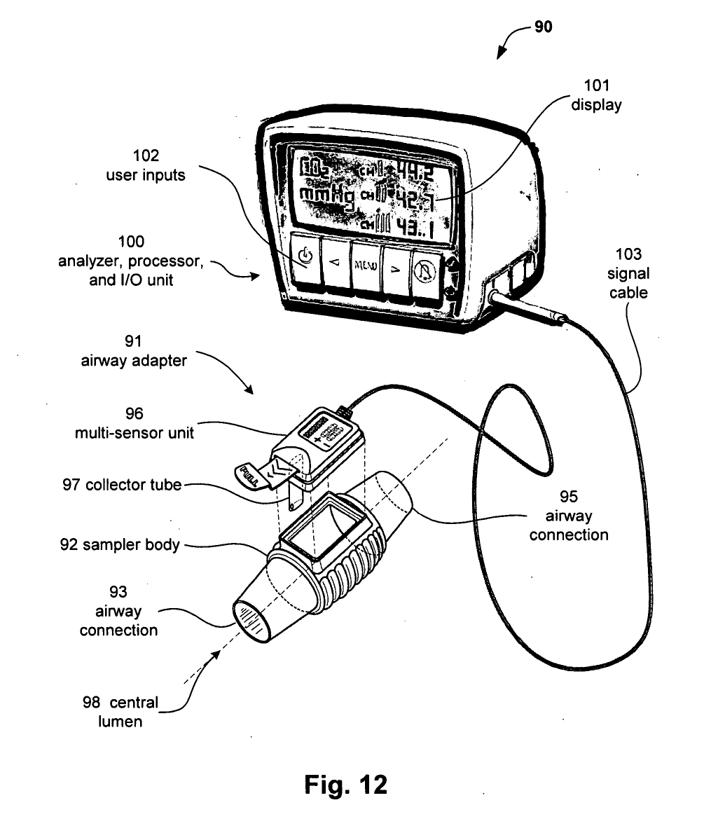

FIG. 12 shows an exemplary integrated multi-analyte breath analysis system 90 having aspects of the invention. As a general description of the layout of this example embodiment, the system 90 comprises a breath sampler 91 and an analyzer-processor-I/O unit 100 communicating with the sampler 91 by signal cable 103. Sampler 91 includes a sampler body 92 having a central lumen 98 in communication with airway connectors 93 and 95.

In operation, air fed into the central lumen 98 from an airway and conducted to collector tube 97. At least one and preferably a plurality of breath constituent species are measured by one or more sensors such as are described herein. In this example the sensor or sensors are mounted in a detachable multi-sensor unit 96, which is shown communicating with central lumen 98 via collector tube 97. One or more measurement signals are transmitted by the multi-sensor unit 96 vial signal cable 103 to analyzer-processor-I/O unit 100. The airway (not shown) in communication with airway connectors 93 and 95 may carry either or both of inspired breath and exhaled breath, depending on the medical application.

The example of sampler 91 shown has the advantage that the sensors of detachable multi-sensor unit 96 are arranged to minimize measurement time lag and dead-space, while conveniently permitting either sensors or the airway adapter to be replaced, as needed. The arrangement provides a high degree of operational flexibility to respond to the sometimes competing needs of low cost, simplicity, avoidance of contamination, and maintaining sensor accuracy.

Note that the breath flow geometry shown in FIG. 12 is but one example having aspects of the invention, and alternative flow arrangements are possible without departing from the spirit of the invention. For example, inspiration and exhalation breath may be combined in a single airway portion in alternation, or inspiration and exhalation may be via separate airways, e.g., controlled by valves. Collector tube 97 may be adapted to sample breath flowing in either or both directions in lumen 98. See, for example, the various alternative sensor/airway configurations described in the above incorporated U.S. Ser. No. 11/019,792 and U.S. Ser. No. 11/488,456.

Optional components (not shown) may be included, such as filters, valves, backflow preventors, mass flow controllers, flow velocity sensors, treatment agent injectors, and the like. As appropriate, such optional components may communicate with and be monitored or controlled via unit 100. Likewise, alternative sensor arrangements are possible without departing from the spirit of the invention. For example, sensors could alternative be mounted apart from sampler 91, for example in analyzer unit 100, communication with sampler 91 via extended air sample tubes (not shown). In another alternative, sensors may be mounted within a mouthpiece, or in an extension tube within the patients mouth or throat, in communication with unit 100.

Analyzer-processor-I/O unit 100 preferably includes at least one display 101 or other output device for communicating with a patient or operator (an LCD display is shown), and also preferably includes at least one user input device 102 (several buttons are shown) to permit convenient patient inputs. In addition, analyzer-processor-I/O unit 100 may include conventional components, such as power supplies, batteries, cable connectors, and the like, common to consumer operated electronic devices. The Analyzer-processor-I/O unit 100 preferably includes signal analyzer to maximize the medical utility and relevance of the measurements of multi-sensor unit 96, as well as memory components to maintain a measurement history. In certain alternatives, the Analyzer-processor-I/O unit 100 may include circuitry to provide wireless and/or internet connectivity, for example to permit medical practitioner to monitor patient-specific measurements remotely, to remotely program the processor/memory to change the measurement routines and parameters in light of patient measurements, to transmit advice re responsive medication dosages, and the like.

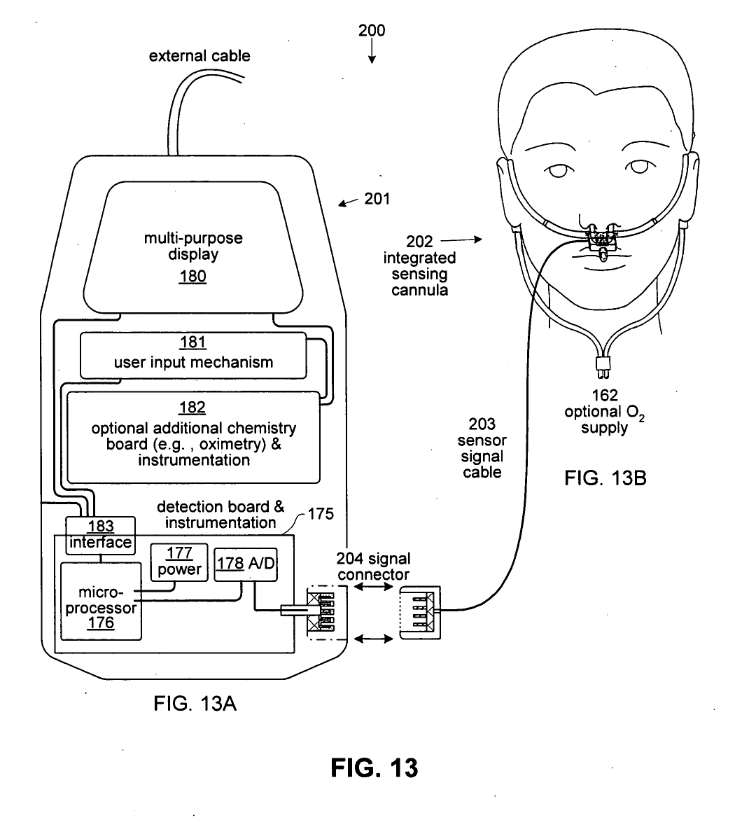

FIG. 13 is a diagrammatic depiction of an exemplary configuration of a portable medical gas sensing system 200, including two linked portions: FIG. 13A depicting a processor unit 201, and FIG. 12B showing integrated sensing cannula 202 worn by a patient, optionally connected to supplemental oxygen (O2) source 162. Note that the figures includes both surface and internal elements, diagramed to clarify functional relationships rather than show realistic physical appearance.

Preferred embodiments may optionally include other complementary chemistry measurements relevant to the patient's care in addition to breath analytes, such as pulse oximetry and the like. The exemplary embodiment of FIG. 13 is shown integrated with a portable oximetry system, for example, such as the 920M™ PLUS and 9600 Avant™ pulse oximetry systems by Respironics, Inc., of Murrysville, Pa.; the Rad-5™ and Rad-57™ pulse oximetry systems by Masimo Corporation, Irvine, Calif.; or the OxiMax® NPB-40 and OxiMax® NPB-75 pulse oximetry systems by Nellcor Puritan Bennett, Inc., Pleasanton, Calif. Additional components for such other analyte systems (e.g., finger-mounted adaptors for oxymetry and the like) are not shown.

As shown in FIG. 13, the sensing system 200 comprises breath sampling cannula 161, optionally connected to supplemental oxygen (O2) source 162. Sensing cannula 202 in turn communicates with portable processor-instrumentation-interface-input-display unit (“processor unit”) 201 by means of sensor signal cable 203 and signal connector 204. For manufacturing and servicing convenience, the processor unit 201 preferably includes a dedicated breath sensing or capnography board 175. In a certain embodiments, board 175 (and/or equivalent distributed components), includes a microprocessor 176 and power supply 177. Signals transmitted from cannula 202 in response to a breath sample pass (e.g., through A/D converter 178) to microprocessor 176 by suitable transmission circuitry. The microprocessor 176 may use the coefficients to calibrate the sensor signal so as to accurately reflect concentration of an analyte of interest, such as CO2 and/or anesthesia agents, in the breath sample. In certain embodiments, the microprocessor 176 may be configured to determine a breathing cycle of the patient (either from measured sensor signals or other detectors), and select sensor signals corresponding to particular portions of the patient's breathing cycle.

The microprocessor 176 is shown in communication via interface connector 179 with display unit 180 and user input mechanism 181. For example, display unit 180 may include an LCD or other display for output of sensor measurements, user-specific configurations, complementary oximetry results from oximetry board 182, and the like. User input mechanism 181 may include a plurality of dedicated buttons and/or a “generic” keypad to permit user control, programming and configuration. Optional user interface elements (not shown) may also include auditory or light outputs and alarms, and may include auditory or light inputs, such as voice command recognition, IR data downloading, and the like.

The microprocessor 176 preferably communicates via an external connector 183 to permit transfer of data to and/or from external sources, such as remote monitoring and recording units. Alternatively, unit 201 may include wireless or RF communication elements (not shown) in communication with microprocessor 176 so as to permit external data exchange.

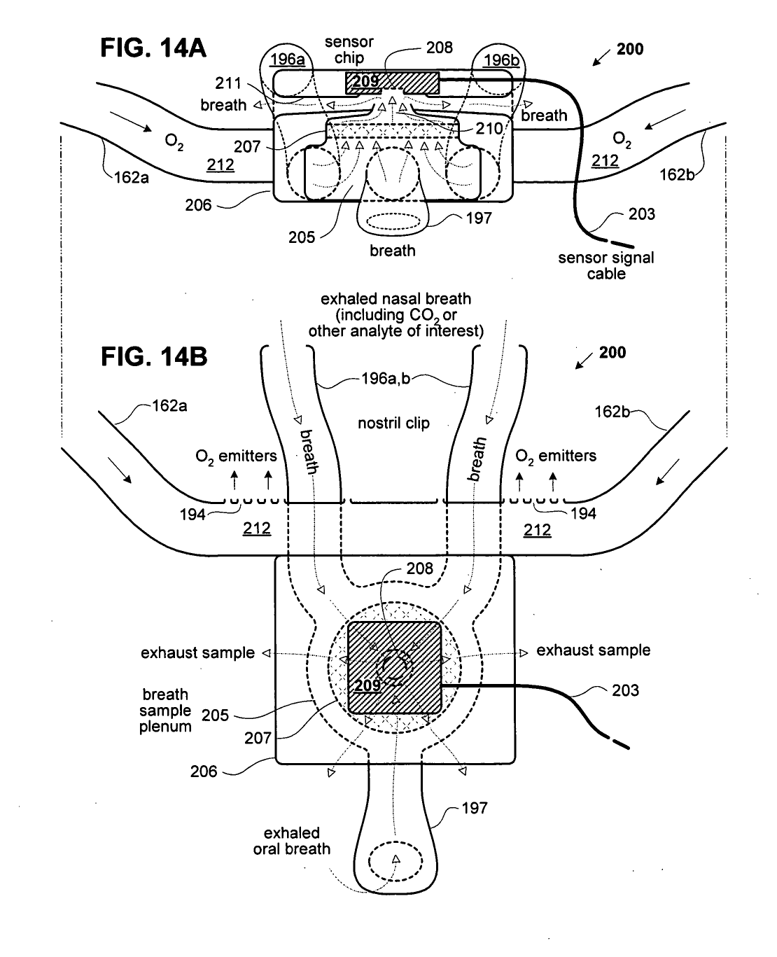

FIGS. 14A and 14B are a cross-section and top view respectively of the integrated CO2 sensing, O2 delivery cannula 202, included in FIG. 13. In operation, during patient exhalation, one or both of a nasal breath sample or an oral breath sample enters the sensing cannula 202, containing a variety of analytes of potential medical interest, such as CO2 and anesthetic agents. The velocity and pressure of the exhaled nasal breath causes a sample to enter nostril tubes 196a,b and pass into central sample plenum 205 within cannula body 206. Similarly, the velocity and pressure of exhaled oral breath causes a sample to enter oral tube 197 and pass into central sample plenum 205 to mix with the nasal sample. The mixed breath sample then flows through optional preconditioner 207, housed adjacent to and communicating with plenum 205. As in the previous example, pre-conditioner or filter (“filter”) 207 may include one or more of absorbents, filters, semi-permeable membranes, and the like, to precondition the breath sample prior to sensor contact.

The breath sample next conducted to impinge or contact opening 208 in sensor chip package 209, in this example, the breath being guided and directed by nozzle 210. The sensor chip package 209 may be configured as in the examples described elsewhere in the present application, or in the references incorporated herein, and includes one or more sensors configured to respond to at least one analyte of interest, so as to produce at least one sensor signal. The sensor signal is transmitted to processor unit 201 via signal cable 203 (note that alternative embodiments may include wireless transmission elements, not shown, to pass the sensor signal to the processor unit 201). The sensor chip package 209 may include an array of sensors and may include sensors specific to different analytes, as described above and in the incorporated references. Following sensor contact, the exhaled breath sample flows through annular space 211 to exhaust through the sides of cannula body 206.

In the particular embodiment shown in FIGS. 14A and 14B, the cannula 202 includes an oxygen plenum 212 fed by dual supply tube 162a,b from the left and right sides of patient. A plurality of oxygen emitters 194 add supplemental oxygen to the inhaled air adjacent patients nostril entrance.

Note that the incorporation of sensor package 209 in cannula 202 provides a number of important advantages. The internal “dead volume” of the measurement equipment is dramatically reduced, since there is no need to convey the breath sample to a remote sensor. In addition, the close proximity of the sensor package 209 to the patients nostrils and mouth assure that there is minimal time delay between exhalation and sensor contact. Nanoelectronic sensors having aspects of the invention are suitable for large scale, inexpensive production, making it feasible to products cannula 202 (and optionally with 203 and connector 204) as a pre-sterilized disposable unit.

Alternative Capacitive Nanosensors.

FIG. 15, views (a) and (b), show an exemplary sensor device, configured as a planar (2D) embodiment of a CNT network capacitance sensor 40, as further described in commonly assigned U.S. Provisional Application No. 60/669,126 filed Apr. 6, 2005; No. 60/683,460 filed May 19, 2005; and No. 60/773,138 filed Feb. 13, 2006; each of which is incorporated by reference. View (b) is a detail portion shown in a magnified sub-drawing at the left. The sensor device 40 comprises a nanostructured film or network 41, preferably including an interlinking network of carbon nanotubes disposed on a substrate 42. The substrate may be generally similar to that described for other embodiments herein, e.g. a silicon base with a dielectric top layer, e.g. SiO2. The nanotube network may be formed as described herein. The network 41 may be functionalized to suit a particular application and target analyte or analytes. In the example shown, at least two conducting contacts 44a and 44b are included, e.g., formed by metal vapor deposition and masking so as to be arranged in an interdigitated fashion upon the nanotube network 41.

Note that a defined portion of the nanotube network is selectively burned, etched or otherwise removed from a patterned offset (e.g., using appropriate masking or the like), so that one of the contact sets 44a (when deposited) lies free of contact with the remaining network 41, and the other contact 44b set lies in electrical contact or communication with the network. In the example shown in FIG. 16, the space between interdigitated fingers of contacts 44a and 44b generally includes a width “d” of network adjoining a gap “g” of bare substrate, so as to form the elements of a capacitor. Upon application of a voltage potential between contacts 44a and 44b, charge accumulates on the spaced-apart contact 44a and the nanotube network 41, separated by gap “g”, thereby producing an electric field potential between the two. Preferably, the offset gap “g” is small. Interaction of an analyte of interest (not-shown) with the nanotubes of the network 41 will tend to change the effective dielectric of the gap, and thus measurably change the capacitance (particularly in the case of species with a substantial dipole). The nanotube network (or other nanostructure) provides a large number of small features which act to intensify the electric field gradient locally, increasing signal-to-noise ration of a signal in response to an analyte of interest.

FIG. 16 is a plan view, cross-sectional view, and equivalent circuit diagram of an exemplary capacitive nanosensor embodiment 10 having aspects of the invention, comprising a bi-layer architecture including a substrate 11 (e.g., PET) and a conductive base or plate 12 (e.g., metal such as Au, graphite, and the like). A dielectric layer 13 (e.g., a polymer, SiO2, and the like, or combinations thereof is interposed between base plate 12 and a nanostructured element 14 (such as one or more CNT or a CNT network). Nanostructured element 14 is capacitively coupled to conductive base 12 in that base 12 is space apart from element 14 to form a pair of capacitive plates. Digitated top lead 15 is shown contacting CNT element 14 to permit electrical communication with measurement circuitry (not shown). Preferably, top leads 15 are applied in such a manner as to prevent contact with base plate 12, so as to avoid a current path between a capacitive plate pair 12, 14, as shown in equivalent circuit 16.

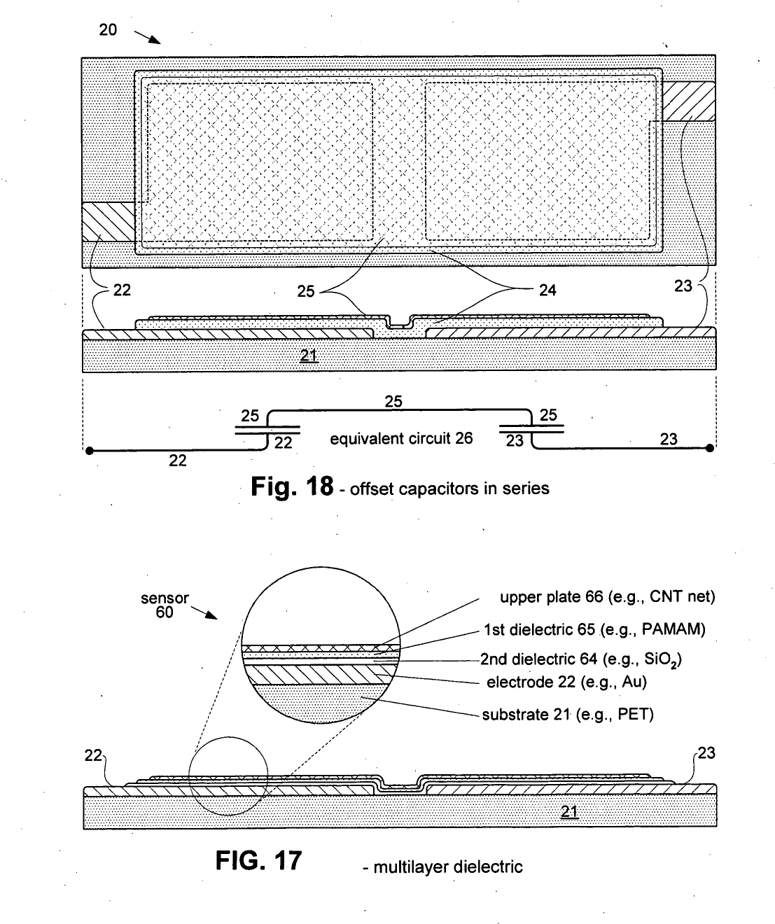

FIG. 17 is a plan view, cross-sectional view, and equivalent circuit diagram of an alternative exemplary capacitive nanosensor embodiment 20 having aspects of the invention, comprising off-set capacitor elements in series, including a substrate 21 (e.g., PET) and an offset pair of conductive leads 22, 23 (e.g., metal such as Au, graphite, and the like), preferably disposed side-by-side adjacent substrate 21, separated by a selected gap. Dielectric layer 24 (e.g., a polymer, SiO2, and the like, or combinations thereof covers active regions of leads 22, 23 and in turn supports CNT element 25, such as a carbon nanotube network. Advantageously, CNT element 25 forms a common capacitive plate electrode opposing both leads 22 and 23 (capacitively coupled), as shown in equivalent circuit 26.

FIG. 18 is a cross-sectional view and a magnified portion of an exemplary capacitive nanosensor embodiment 60 having aspects of the invention, generally similar to that shown in FIG. 17 (see elements 21, 22, and 23) and having a multi-layer dielectric structure comprising first dielectric layer 64 and a second dielectric layer 65. One of more of layers 64 and 65 are interposed between leads 22, 23 and CNT element 66. For example, layer 64 may comprise a porous or non-porous material such as SiO2, and layer 65 may comprise a polymer, such as porous PAMAM. Both the porosity and hydrophilicity/hydrophobicity as well a other properties of layers 64 and 65 may be selected to suit a particular application, analyte medium and the like. One of layers 64 or 65, or an additional layer, may lie above or embedding CNT layer 66.

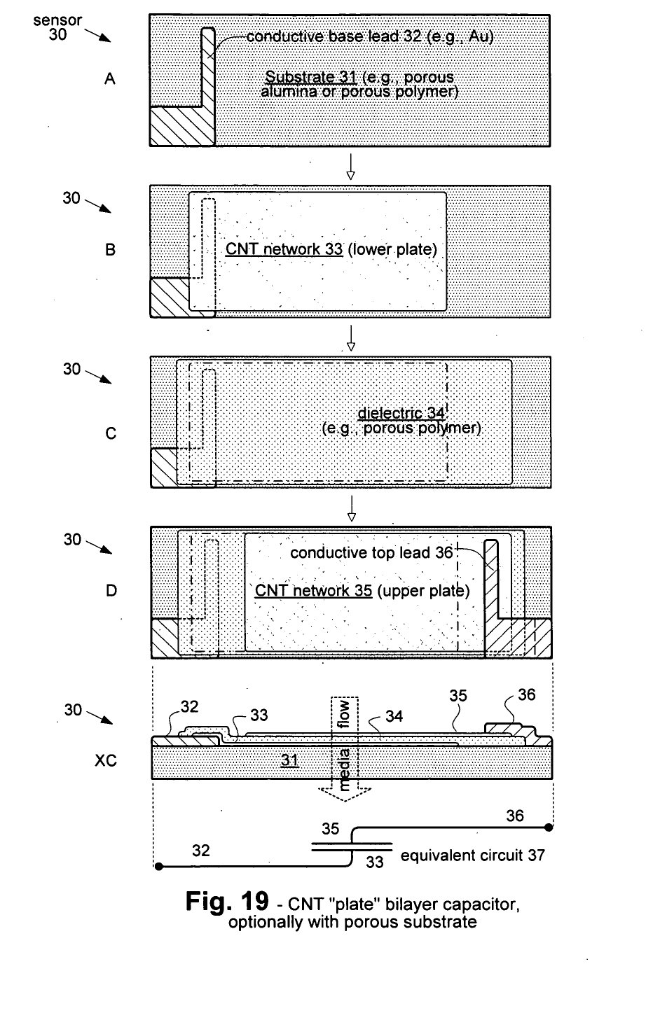

FIG. 19 is a schematic and equivalent circuit diagram which illustrates an exemplary capacitive nanosensor embodiment 30 having aspects of the invention, and having a bi-layered architecture comprising a first base lead or contact pad 32 disposed adjacent a substrate 31 (porous in this example, such as porous alumina). Lead 32 contacts a lower CNT plate or element 33, which is preferably shaped so as to have an active region 37 off-set from contact 32. At least the active region of plate 33 is covered by dielectric layer 34 (e.g., porous polymer or inorganic material such as SiO2). Upper CNT plate 35 covers at least the active region of lower plate 33, electrically isolated by dielectric 34, and is in turn contacted by a top lead or contact 36, which is likewise preferably offset from the active region 37. Thus, in FIG. 19, upper plate 35 is adjacent lower plate 33 and in electrical contact with lead 36, which is in turn offset and removed from proximity to plate 33. Analyte media may advantageously flow perpendicularly to substrate 31, and the upper and lower plates 33,35 form a capacitive plate pair removed from leads 32, 36, as shown in equivalent circuit 37.

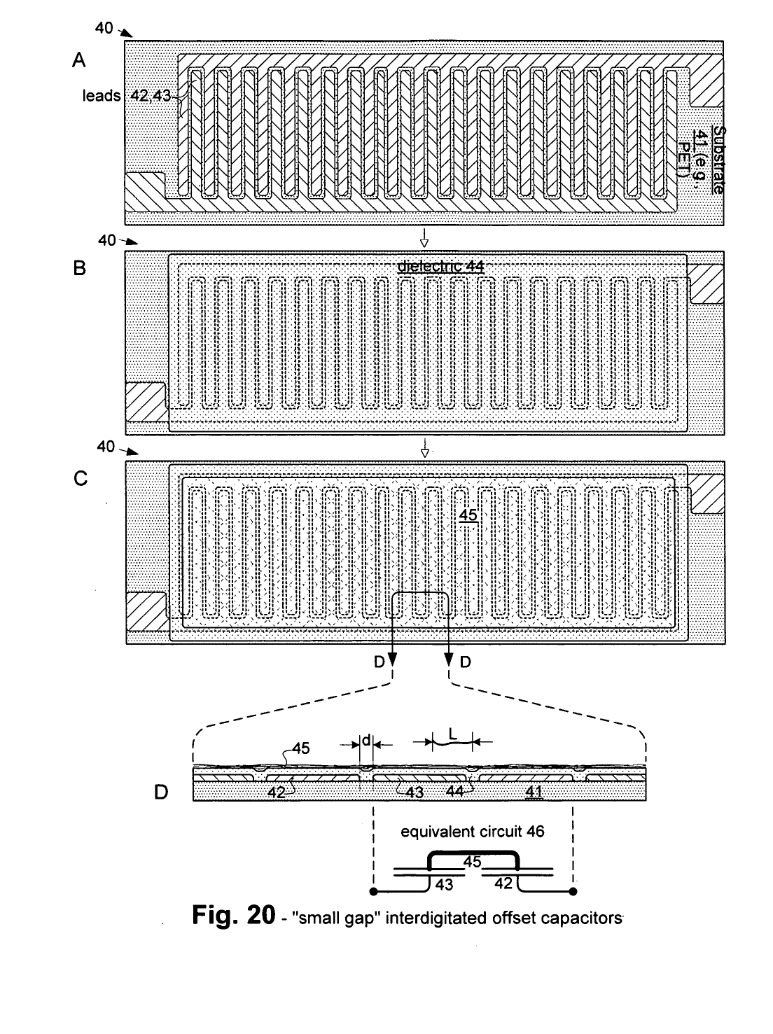

FIG. 20 is a schematic diagram and equivalent circuit which illustrates an exemplary capacitive nanosensor embodiment 40 having aspects of the invention. In schematic architecture, the sensor 40 is similar in a number of respects to that for FIG. 17, in that conductive leads 42, 43 (e.g., metal such as Au, graphite, and the like), form an offset pattern adjacent substrate 41, covered by dielectric 44 and CNT element 45. In this example, leads 42, 43 are arranged so as to have a characteristic gap “d” that is small in comparison to the typical or characteristic length “L” of the nanotubes comprising CNT element 45 (which may include one or more aligned CNTs, or may comprise a random network). Note that while neither the gap nor the CNT length need be uniform, the statistical effect of the relation of the characteristic dimensions is that substantial numbers of nanotubes span the gap so as to have a portion capacitively coupled to each conductive lead. Advantageously, conductive leads 42, 43 may be arranged in an interdigitated pattern, and gap “g” may be created by conventional lithographic deposition methods, or may selectively etched in a continuous material. The continuity of conduction within CNT network 45 provides a low resistance path connecting the “series capacitor” regions adjacent leads 42, 43, as shown in equivalent circuit 46.

As may be seen in the foregoing examples, devices having aspects of the invention may be configured to exploit the electrical properties of one or more nanostructures, such as a film or network of nanotubes, without direct contact of conductive circuit elements with the nanostructures (e.g., without metal-to-nanotube contact regions).

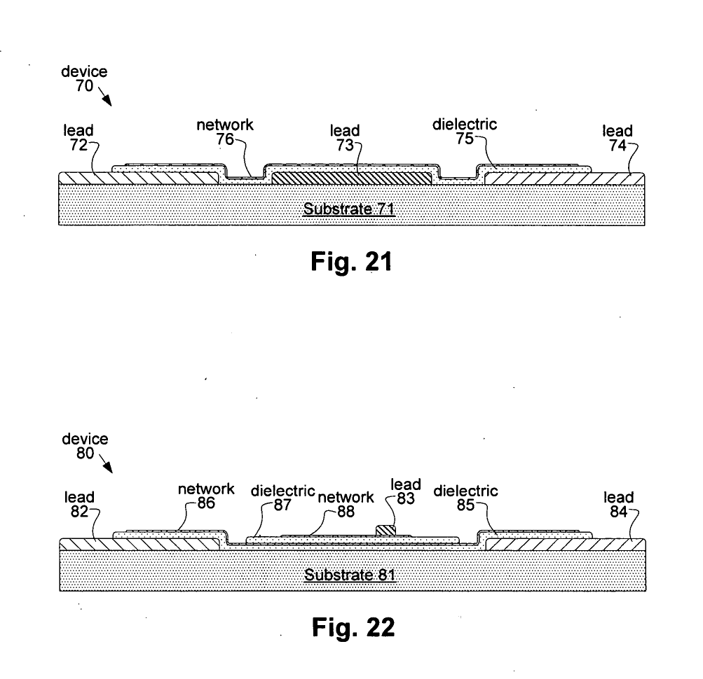

FIGS. 21 and 22 are cross-sectional views showing exemplary nanostructured devices having a network element such as a CNT network which is electrically coupled to multiple leads without direct lead-to-network contact. For example, in FIG. 21, device 70 (such as a nanosensor) comprises electrically continuous network 76 (such as a CNT film of greater density than the percolation limit) which is separated from spaced apart leads 72, 73 and 74 by dielectric layer 75, permitting each such lead or electrode to be capacitively coupled to network 76 without direct contact (e.g., avoiding metal-to-CNT contact). In an alternative example, in FIG. 22, device 80 (such as a nanosensor) comprises electrically continuous network 86 which is separated from spaced apart leads 82 and 84 by dielectric layer 85, permitting these leads to be capacitively coupled to network 86 without direct contact.

An additional electrical influence on network 86 comprises a second plate-like network element 88, which disposed over network 86 and separated from network 86 by an additional dielectric region 87. The “plate” network is shown contacting a third lead 83, although it should be understood that lead 83 may be physically remote or offset from the network 86, such as by the arrangement shown in FIG. 19 (in FIG. 19, upper plate 35 is adjacent lower plate 33 and in electrical contact with lead 36, which is offset and removed from proximity to plate 33). The application of DC and/or AC voltages of selected frequency ranges to the leads (e.g., AC with DC bias) can result in selected electrical influences, responsive to the electrical properties of the nanotubes (e.g., resistance, impedance, inductance, capacitance, or combinations of these, and the like). The dimensions and properties of the various elements can be selected by one of ordinary skill in the art to provide desired device properties, such as high-pass, low-pass filter effects, of the various subassemblies and components.

Method of Dynamic Sensor Sampling.

In one inventive aspect, a method of dynamic sensor sampling is provided, which permits measurement of analyte concentration over time, while avoiding exposure of the sensor to a sample medium on a continuous basis. For example, a valve or fluidic circulation system may be included to selectively expose a sensor having aspects of the invention to a sample medium. In certain embodiments, a dynamic sampling method permits minimizing exposure of a sensor to corrosive or life-limiting environmental conditions. In other embodiments (e.g., and electrochemical sensor), a dynamic sampling method may conserve reagent supply and extend service life. In yet other embodiments, a dynamic sampling method may avoid irreversible or persistent changes in sensor properties. In still other embodiments a dynamic sampling method may permit more rapid sensor response to changes in analyte conditions and reduce recovery time. A dynamic sampling method may also be employed to reduce cross-sensitivity, where response to a cross-reactant is slower than to a target analyte.

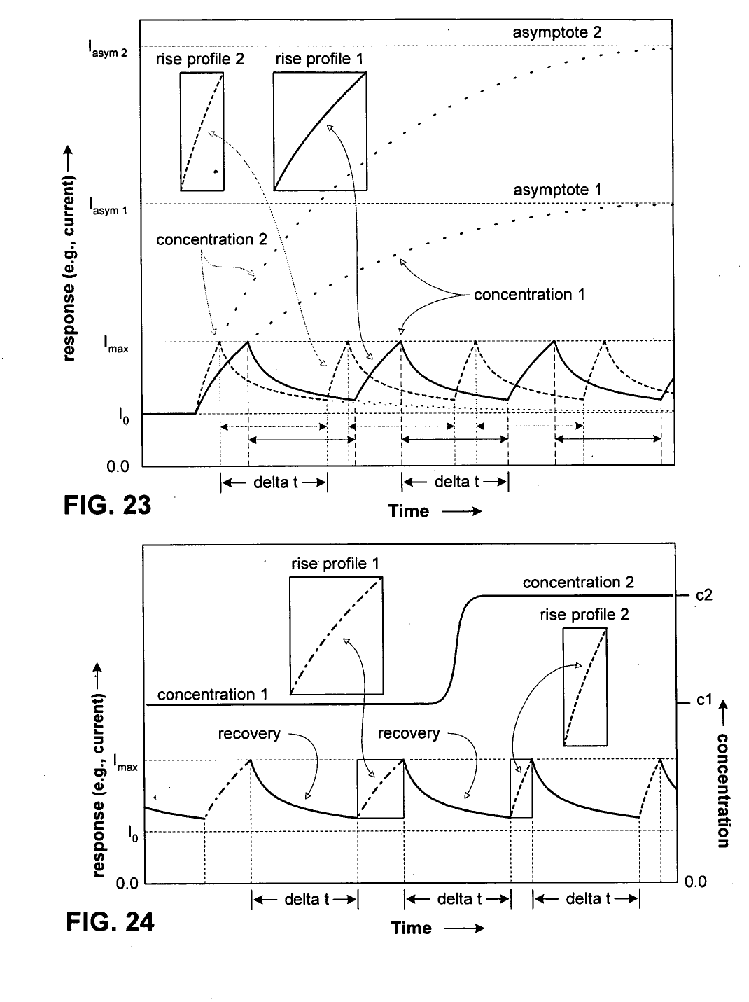

FIG. 23 is a schematic plot illustrating principles of a dynamic sensor sampling method having aspects of the invention. The vertical axis represents a nominal sensor response magnitude. In the example shown, this is an electrical current I (e.g., across a channel element of a transconductance sensor) but the response may represent any one of a number different sensor properties, such as a conductance, resistance, capacitance, impedance or the like. The response may also represent a complex or derived property, such as a ratio, modulation, time constant, exponent or other relationship associated with measured properties. The response may alternatively represent a statistical property in relation to multiple sensors of a sensor array, such as a mean value or the like.

As may be seen in FIG. 23, the unexposed sensor is initially at an response level (I₀). Exposure of the sensor to a first analyte concentration (concentration 1) produces a sensor response that increases over time so as to asymptotically approach (dotted curve) a steady-state response magnitude (I_{asym1}). If the sensor is isolated from exposure to a sample (or otherwise prevented from responding to an analyte, such as by a controllable inhibitor) at a point when the response reaches a selected cut-off magnitude (Iₘₐₓ), a recovery trend is begun, the response value declining so as to asymptotically approach the initial value I₀. If the sensor is again exposed to the analyte sample after a recovery interval (delta t), the sensor response again increases (“rise profile”) in a similar manner until the cut-off value Iₘₐₓ is reached.

A second curve in FIG. 23 represents the response of the sensor to an analyte sample of a differing concentration (heavy dashed line—concentration 2), such that the response that increases over time so as to asymptotically approach (dotted curve) a different steady-state response magnitude (I_{asym2}). If the exposure is interrupted at a cut-off value (Iₘₐₓ), and the sensor is permitted to recover for a selected interval (delta t), the response curve of concentration 2 is similar to that of concentration 1, but having a differing rise profile (rise profile 1 vs. rise profile 2). Analytical comparison of the rise profiles may be employed to characterized the analyte concentrations, without monitoring the sensor response until a steady-state response magnitude is reached or approached.

FIG. 24 is a schematic plot an example of dynamic sensor sampling for a step change in analyte concentration. As in FIG. 23, the sampling method in this example applies a fixed maximum response cut-off value Iₘₐₓ and a fixed recovery interval delta t. The curve of sensor response shows a change in rise profile following the change in analyte concentration (rise profile 1 vs. rise profile 2). It should be understood that in the example shown, the sensor recovery is consistent, independent of analyte concentration, and approaches (I₀) without a persistent off-set. However, this may not be so, and methods of dynamic sampling may be applied effectively to sensors which do not exhibit these characteristics. For example, accumulated drift in sensor response may be compensated for. A number of alternative analytical algorithms may be applied to correlate rise profile with analyte concentration.

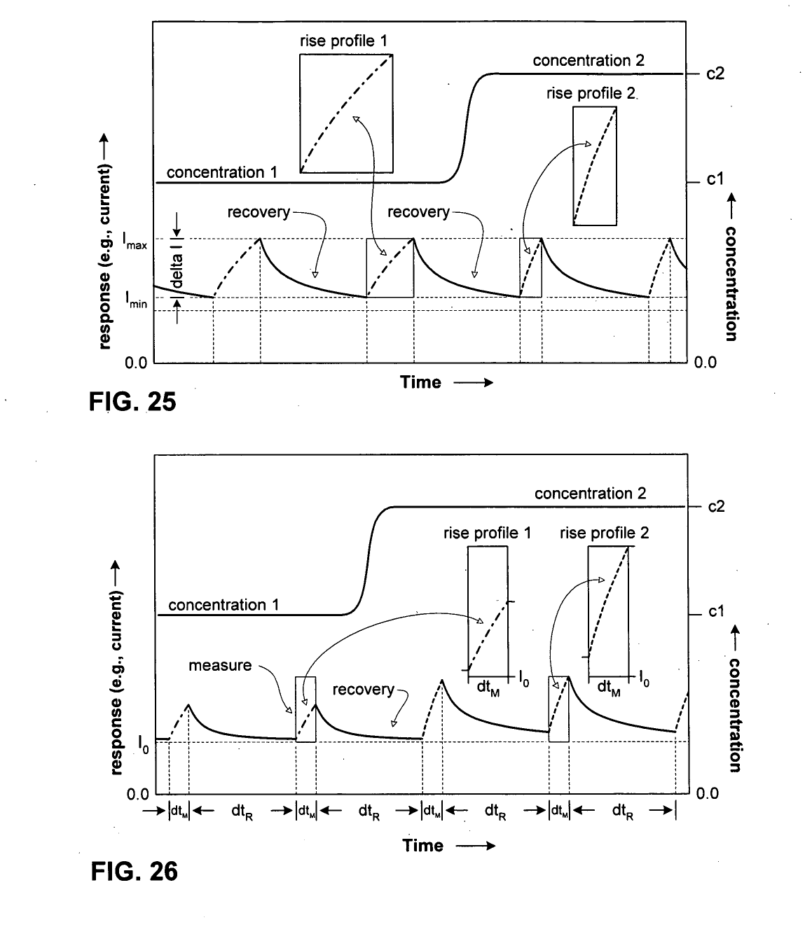

FIG. 25 is a schematic plot an alternative example of dynamic sensor sampling for a step change in analyte concentration, having both fixed maximum and minimum response cut-off values. As may be seen, the measurement and recovery phases (analyte exposure and isolation) are triggered by a response magnitude reached a maximum and minimum value (Iₘₐₓ and Iₘᵢₙ).

FIG. 26 is a schematic plot an example of dynamic sensor sampling for a step change in analyte concentration, having a both fixed measurement and recovery intervals. As may be seen, the measurement and recovery phases are triggered by the passage of a determined measurement interval (dt_{M}) and recovery interval (dt_{R}).

I should be understood that a sensor system may employ the sampling modes of FIGS. 24-26 alone, in sequence or in combination. For example, a sensor system may be programmed to apply a certain sampling mode for analyte concentrations in a certain range and another sampling mode for another range of analyte concentrations for a stand-by or active mode, or the like. Additional alternative modes of sampling may be employed without departing from the spirit of the invention.

In like fashion to that described in the example above, alternative functionalization materials and alternative device architectures may be included (e.g., alternative electrode elements and nanostructures, such as nanowires, MWNTs, non-carbon or hetero nanotubes other known nanoparticles, and the like). Such alternatives may include measurements of other device properties, such as capacitance, impedance and the like.

Having thus described a preferred embodiment of nanostructures with electrodeposited nanoparticles, and methods of making them, it should be apparent to those skilled in the art that certain advantages of the within system have been achieved. It should also be appreciated that various modifications, adaptations, and alternative embodiments thereof may be made within the scope and spirit of the present invention. For example, specific examples have been illustrated for nanotube film nanostructures, but it should be apparent that the inventive concepts described above would be equally applicable to other types of nanostructures. In addition, the sensor devices having aspects of the invention may be adapted or employed to detect or measure other organic and inorganic compounds. For example, various ones of the embodiments described may be adapted for measurement or detection of vapor species associated with organic explosive compositions. The invention is further defined by the following claims.

1. A sensor for detecting an analyte in a sample, comprising:
a substrate; and
a capacitance circuit disposed adjacent the substrate, the circuit including at least a first capacitive element configured to interact with a sample, wherein the circuit is configured to respond to the presence of an analyte of interest by a measurable change in an electrical property.

2. The sensor of claim 1, wherein the first capacitive element comprises a conductive nanostructured material.

3. The sensor of claim 2, wherein the nanostructured material comprising the first capacitive element includes an interconnecting network of carbon nanotubes.

4. The sensor of claim 3, further comprising a second capacitive element spaced apart from the first capacitive element

5. The sensor of claim 4, wherein the second capacitive element comprises a nanostructured material.

6. The sensor of claim 4, wherein the second capacitive element is isolated from the first capacitive element by at least a layer of dielectric material.

7. The sensor of claim 6, further comprising one or more electrodes in communication with the first capacitive element and configured to measure at least one transconductance property of the first capacitive element.

8. The sensor of claim 7, further comprising a gate electrode, the gate electrode configured to influence a transconductance property of the first capacitive element.

9. The sensor of claim 4, further comprising a processor in communication with the circuit and configured to measure both a capacitance property of the circuit and a transconductance property of the first capacitive element, wherein the processor is further configured to determine one of the presence and the concentration of the analyte by determining a relationship between the change in the capacitance property and the transconductance property in response to exposure to the sample.

10. The sensor of claim 3, wherein the nanostructured material comprising the second capacitive element includes an interconnecting network of carbon nanotubes.

11. The sensor of claim 1, wherein the circuit is configured to have a sensitivity to at least one of an organic compound or an inorganic compound.

12. The sensor of claim 11, wherein the organic compound includes a halogenated anesthetic agent and the inorganic compound includes nitrous oxide.

13. The sensor of claim 11, wherein the organic compound includes a vapor species associated with an organic explosive composition.

14. The sensor of claim 1, further comprising a layer associated with the first capacitive element and configured to inhibit the exposure of the first capacitive element to at least one species from the sample.

15. The sensor of claim 1, further comprising a recognition material associated with the first capacitive element and configured to influence the electrical property.

16. A sensor for detecting an analyte in a sample, comprising:
a substrate; and
a circuit structure including:
a first electrically active element; and
at least a second electrically active element spaced apart from the first electrically active element and configured to influence at least one electrical property of the first electrically active element, and
a processor configured to measure the at least one electrical property upon exposure of the sensor to the sample, and configured to determine a change in the at least one electrical property in response to the analyte;
wherein one or both of the first and second electrically active elements comprises a conductive nanostructured material.

17. The sensor of claim 16, wherein the influence of the second electrically active element on the first electrically active element includes one or more of:
(a) a coupling influencing a capacitance property of the circuit structure;
(b) a field emission effect influencing one of a breakdown voltage or an electron flow relative to the first electrically active element;
(c) an electrochemical effect influencing a current flow relative to the first electrically active element;
(d) a field influence on a transconductance property of the first electrically active element.

18. The sensor of claim 16, wherein the nanostructured material comprising at least one of the first and second electrically active elements includes an interconnecting network of carbon nanotubes.

19. The sensor of claim 16, wherein the circuit structure is configured to have a sensitivity to at least an organic compound.

20. The sensor of claim 19, wherein the organic compound includes a halogenated anesthetic agent.

21. The sensor of claim 19, wherein the organic compound includes a vapor species associated with an organic explosive composition.

22. A breath analyzer system comprising:
a breath sampling cannula including one or more lumens configured to by mounted adjacent at least one of a patient's nostril and mouth, the lumen having an opening arranged to gather an exhaled breath sample upon patient exhalation;
one or more nanostructure sensor comprised as in claim 1, the sensor in communication with the lumen of the breath sampling cannula, so as to contact at least a portion of the exhaled breath sample; the sensor having a sensitivity to an anesthetic agent in human exhaled breath so at to produce a sensor signal in response to the anesthetic agent;
a processing unit in communication with the sensor so as to receive the sensor signal, the processor unit configured to use the signal to determine a measurement of one of: (i) the concentration of anesthetic agent in the sample; and (ii) the amount of anesthetic agent in the sample, and
an output device in communication with the processing unit and configured to output at least the measurement to a user, so as to provide information related to a human medical state.

23. A breath analyzer system comprising:
a breath sampling cannula including one or more lumens configured to by mounted adjacent at least one of a patient's nostril and mouth, the lumen having an opening arranged to gather an exhaled breath sample upon patient exhalation;
one or more nanostructure sensor comprised as in claim 16, the sensor in communication with the lumen of the breath sampling cannula, so as to contact at least a portion of the exhaled breath sample; the sensor having a sensitivity to an anesthetic agent in human exhaled breath so at to produce a sensor signal in response to the anesthetic agent;
a processing unit in communication with the sensor so as to receive the sensor signal, the processor unit configured to use the signal to determine a measurement of one of: (i) the concentration of anesthetic agent in the sample; and (ii) the amount of anesthetic agent in the sample, and
an output device in communication with the processing unit and configured to output at least the measurement to a user, so as to provide information related to a human medical state.

24. A sensor, comprising:
a substrate;
a spaced-apart pair including a first conductive lead and a second conductive lead disposed adjacent the substrate;
a dielectric material covering at least a region of at least one conductive lead; and
one or more nanostructures disposed adjacent the dielectric material and capacitively coupled to at least one conductive lead.

25. The sensor of claim 24; wherein the one or more nanostructures comprises an electrically-continuous network including a plurality of carbon nanotubes spanning to cover at least a region of each conductive lead, dielectric material disposed to isolate the network from each of the first and second conductive leads.

26. The sensor of claim 25; wherein the spaced-apart pair of conductive leads have a characteristic separation gap “g”, and wherein the carbon nanotubes have a characteristic length “L”, and wherein “L” is significantly greater that “g”.

27. The sensor of claim 25; wherein substantial numbers of nanotubes span the gap so as to have at least a portion of the spanning nanotube capacitively coupled to the first lead and at least a portion of the spanning nanotube capacitively coupled to the second lead.

28. The sensor of claim 24; further comprising a functionalization material disposed adjacent the carbon nanotubes.

29. The sensor of claim 24; wherein the dielectric material comprises a plurality of layers, each layer having a distinct composition.

30. A sensor, comprising:
a substrate having an active region;
first and second conductive leads disposed adjacent the substrate and space apart from the active region;
a dielectric material disposed adjacent at least the active region; and
first and second nanostructure layers in electrical communication with the first and second conductive leads respectively; the nanostructure layers each including one or more nanostructures; the nanostructure layers arranged adjacent the active region and configured so as to be capacitively coupled and separated with respect to each other by the dielectric material.

31. The sensor of claim 30; wherein the one or more of the nanostructure layers comprises a network of carbon nanotubes.

32. The sensor of claim 30; further comprising a functionalization material disposed adjacent the carbon nanotubes.

33. The sensor of claim 30; wherein at least a portion of the substrate and at least a portion of the dielectric material is porous and configured to permit an analyte medium to pass through the substrate active region.

34. A molecular sensor comprising:
a) a nanotube device comprising at least one carbon nanotube, wherein a first end of said nanotube is electrical coupled to a first conducting element without direct contact, and a second end of said nanotube is electrical coupled to a second conducting element; and
b) a coating of one or more sensing agents deposited on said nanotube; wherein said sensing agents are so chosen such that the agents-coated nanotube responds to a particular molecular species.

35. The molecular sensor of claim 34; wherein second end of said nanotube is electrical coupled to a second conducting element without direct contact.

36. A nanotube device comprising:
a nanotube film comprising a plurality of nanotubes and having a first end and a second end; and
first and second electrodes respectively disposed on said first end and said second end of said nanotube film,
wherein the nanotube film is adapted to pass current between the first and second electrodes without direct contact with at least one of the first and second electrodes.

37. The nanotube device of claim 36; wherein the nanotube film is adapted to pass current between the first and second electrodes without direct contact with either of the first and second electrodes.

38. The nanotube device of claim 37; wherein the nanotube film is adapted to pass current in response to an AC voltage wherein an additional DC bias is applied between the first and second electrodes.

39. A method for controlling the operation of a sensor in monitoring an analyte in a sample environment, comprising the steps of:
(a) selectively exposing at least a portion of a sensor to the environment so that the sensor portion is exposed only intermittently; and
(b) dynamically sampling a response signal output from the sensor so as to determine the presence or concentration of the analyte of by analysis of the dynamically sampled signal.

40. The method of claim 39, wherein selectively exposing includes regulating sensor exposure by means of one or both of a fluidic lumen and a valve.

41. The method of claim 39, wherein dynamically sampling includes analysis of the sensor signal limited to one or more of selected ranges of sensor response and selected time intervals of sensor exposure to the environment.

42. The method of claim 41, wherein dynamically sampling includes limiting the analysis of the response signal to response magnitudes below a cut-off maximum.

43. The method of claim 41, wherein selectively exposing includes regulating sensor exposure to provide for non-exposed recovery time periods of a selected fixed duration.

44. The method of claim 43, wherein the non-exposed recovery time periods are of a selected fixed duration.
